(19) United States
(12) Patent Application Publication
(10) Pub. No.: US 2025/0268577 A1
(43) Pub. Date: Aug. 28, 2025

(54) MEDICAL ASSISTANCE APPARATUS, ULTRASOUND ENDOSCOPE, MEDICAL ASSISTANCE METHOD, AND PROGRAM

(71) Applicant: FUJIFILM Corporation, Tokyo (JP)

(72) Inventor: Toshihiro USUDA, Kanagawa (JP)

(73) Assignee: FUJIFILM Corporation, Tokyo (JP)

(21) Appl. No.: 19/198,150

(22) Filed: May 5, 2025

Related U.S. Application Data
(63) Continuation of application No. PCT/JP2023/039522, filed on Nov. 1, 2023.

(30) Foreign Application Priority Data
Nov. 8, 2022 (JP) 2022-178952

(51) Int. Cl. A61B 8/00; A61B 8/12

(52) U.S. Cl. CPC A61B 8/5223; A61B 8/12; A61B 8/463

(57) ABSTRACT

A medical assistance apparatus includes a processor. The processor displays, on a screen, a first ultrasound image among a plurality of ultrasound images in a time series, the ultrasound image showing a target site of observation and satisfying a predetermined condition, and outputs first size information indicating a first size. The first size is a size of the target site of observation shown in the first ultrasound image.

### CROSS-REFERENCE TO RELATED APPLICATIONS

This application is a continuation application of International Application No. PCT/JP2023/039522, filed Nov. 1, 2023, the disclosure of which is incorporated herein by reference in its entirety. Further, this application claims priority from Japanese Patent Application No. 2022-178952, filed Nov. 8, 2022, the disclosure of which is incorporated herein by reference in its entirety.

### BACKGROUND

### 1. Technical Field

The technology of the present disclosure relates to a medical assistance apparatus, an ultrasound endoscope, a medical assistance method, and a program.

### 2. Related Art

WO2020/008743A discloses an acoustic wave diagnostic apparatus provided with a display unit, an operation unit, a measurement position designation accepting unit, an object-of-measurement recognition unit, a measurement algorithm setting unit, and a measurement unit.

In the acoustic wave diagnostic apparatus described in WO2020/008743A, the display unit displays an acquired acoustic wave image. The measurement position designation accepting unit accepts, from a user via the operation unit, a designation of a measurement position in the acoustic wave image displayed on the display unit. The object-of-measurement recognition unit recognizes an object of measurement included in the acoustic wave image within a recognition range determined on the basis of the measurement position received by the measurement position designation accepting unit. The measurement algorithm setting unit sets a measurement algorithm on the basis of the object of measurement recognized by the object-of-measurement recognition unit. The measurement unit takes a measurement of the object of measurement in the acoustic wave image on the basis of the measurement algorithm set by the measurement algorithm setting unit, and causes the display unit to display a measurement result.

WO2020/008746A discloses an acoustic wave diagnostic apparatus that sequentially displays, on a display unit, multiple successive frames of acoustic wave images being taken. The acoustic wave diagnostic apparatus described in WO2020/008746A is provided with an object-of-measurement recognition unit, a measurement algorithm setting unit, and a measurement unit.

In the acoustic wave diagnostic apparatus described in WO2020/008746A, the object-of-measurement recognition unit automatically recognizes an object of measurement included in the acoustic wave image of the current frame being displayed on the display unit. The measurement algorithm setting unit sets a measurement algorithm for the object of measurement recognized by the object-of-measurement recognition unit. The measurement unit measures the object of measurement on the basis of the measurement algorithm set by the measurement algorithm setting unit, and causes the display unit to display a measurement result superimposed on the acoustic wave image of the current frame.

WO2017/104263A discloses an ultrasound observation apparatus in which the positions of two measurement points defined by instruction input from a touch panel are calculated, and then a measurement-to-measurement distance calculation unit calculates the distance between the two measurement points and causes a display unit to display the calculated distance.

### SUMMARY

An embodiment according to the technology of the present disclosure provides a medical assistance apparatus, an ultrasound endoscope, a medical assistance method, and a program that enable a user to ascertain the size of a target site of observation, the user observing an ultrasound image which shows the target site of observation and which satisfies a predetermined condition.

A first aspect according to the technology of the present disclosure is a medical assistance apparatus including a processor configured to: display, on a screen, a first ultrasound image among a plurality of ultrasound images in a time series, the ultrasound image showing a target site of observation and satisfying a predetermined condition; and output first size information indicating a first size, wherein the first size is a size of the target site of observation shown in the first ultrasound image.

A second aspect according to the technology of the present disclosure is the medical assistance apparatus according to the first aspect, wherein the outputting of the first size information includes displaying the first size on the screen.

A third aspect according to the technology of the present disclosure is the medical assistance apparatus according to the first aspect or the second aspect, wherein the predetermined condition includes a condition stipulating an image which is among the plurality of ultrasound images and in which the target site of observation is detected and the size of the detected target site of observation is measured.

A fourth aspect according to the technology of the present disclosure is the medical assistance apparatus according to the first aspect or the second aspect, wherein the predetermined condition includes a condition stipulating an image which is among the plurality of ultrasound images and in which a specific target site of observation is detected among a plurality of target sites of observation and the size of the specific target site of observation is measured.

A fifth aspect according to the technology of the present disclosure is the medical assistance apparatus according to any one of the first to fourth aspects, wherein the processor outputs a plurality of ultrasound images which are among the plurality of ultrasound images and in which the target site of observation is detected and the size of the target site of observation is measured.

A sixth aspect according to the technology of the present disclosure is the medical assistance apparatus according to any one of the first to fifth aspects, wherein the predetermined condition includes a condition stipulating that a length of the target site of observation is equal to or greater than a reference value.

A seventh aspect according to the technology of the present disclosure is the medical assistance apparatus according to any one of the first to sixth aspects, wherein the predetermined condition includes a condition stipulating that a length of the target site of observation is a maximum value or a mode.

An eighth aspect according to the technology of the present disclosure is the medical assistance apparatus according to the sixth aspect or the seventh aspect, wherein if the target site of observation is a vessel, the length is the length in the radial direction of the vessel.

A ninth aspect according to the technology of the present disclosure is the medical assistance apparatus according to any one of the first to seventh aspects, wherein size information indicating the size of the target site of observation is applied to each of the ultrasound images, and the processor outputs the size information applied to the first ultrasound image as the first size information.

A 10th aspect according to the technology of the present disclosure is the medical assistance apparatus according to any one of the first to ninth aspects, wherein the first ultrasound image is an ultrasound image that satisfies the predetermined condition among the ultrasound images in a timespan defined with respect to a selected ultrasound image that is selected from the plurality of ultrasound images according to a given first instruction.

An 11th aspect according to the technology of the present disclosure is the medical assistance apparatus according to the 10th aspect, wherein the selected ultrasound image is a freeze image, and the freeze image is the ultrasound image displayed on the screen in a frozen state according to the first instruction in a situation in which the plurality of ultrasound images are being displayed as a dynamic image on the screen.

A 12th aspect according to the technology of the present disclosure is the medical assistance apparatus according to the 10th aspect or the 11th aspect, wherein the timespan is a timespan going back from the point in time when the selected ultrasound image is obtained.

A 13th aspect according to the technology of the present disclosure is the medical assistance apparatus according to any one of the 10th to 12th aspects, wherein the length of the timespan is defined according to a given second instruction.

A 14th aspect according to the technology of the present disclosure is the medical assistance apparatus according to any one of the first to 13th aspects, wherein the processor outputs second size information indicating a second size, and the second size is a size of the target site of observation shown in a second ultrasound image different from the first ultrasound image among the plurality of ultrasound images.

A 15th aspect according to the technology of the present disclosure is the medical assistance apparatus according to the 14th aspect, wherein size information indicating the size of the target site of observation is applied to each of the ultrasound images, and the processor outputs the size information applied to the second ultrasound image as the second size information.

A 16th aspect according to the technology of the present disclosure is the medical assistance apparatus according to the 14th aspect or the 15 aspect, wherein the outputting of the second size information includes displaying the second size on the screen.

A 17th aspect according to the technology of the present disclosure is the medical assistance apparatus according to any one of the 14th to 16th aspects, wherein the processor outputs the first size information and the second size information in a distinguishable manner.

An 18th aspect according to the technology of the present disclosure is the medical assistance apparatus according to any one of the first to 17th aspects, wherein the predetermined condition includes a condition stipulating an image which is among the plurality of ultrasound images and in which the target site of observation is detected and the size of the target site of observation is measured according to a measurement method appropriate for the detected target site of observation.

A 19th aspect according to the technology of the present disclosure is the medical assistance apparatus according to any one of the first to 17th aspects, wherein the predetermined condition includes a condition stipulating an image which is among the plurality of ultrasound images and in which a specific target site of observation is detected among a plurality of target sites of observation and the size of the specific target site of observation is measured according to a measurement method appropriate for the specific target site of observation.

A 20th aspect according to the technology of the present disclosure is the medical assistance apparatus according to any one of the first to 19th aspects, wherein the processor outputs a plurality of ultrasound images which are among the plurality of ultrasound images and in which the target site of observation is detected and the size of the target site of observation is measured according to a measurement method appropriate for the target site of observation.

A 21st aspect according to the technology of the present disclosure is the medical assistance apparatus according to the 20th aspect, wherein the measurement method includes a first measurement method and/or a second measurement method, the first measurement method is a method for measuring the target site of observation in one direction, and the second measurement method is a method for measuring the target site of observation in multiple directions.

A 22nd aspect according to the technology of the present disclosure is the medical assistance apparatus according to any one of the first to 21st aspects, wherein the first size is a size of a range corresponding to the target site of observation.

A 23rd aspect according to the technology of the present disclosure is the medical assistance apparatus according to the 22nd aspect, wherein calipers defining the range are displayed on the screen.

A 24th aspect according to the technology of the present disclosure is the medical assistance apparatus according to the 23rd aspect, wherein a geometric property of the calipers is changed according to a given third instruction, and the range is changed in association with the change in the geometric property.

A 25th aspect according to the technology of the present disclosure is The medical assistance apparatus according to any one of the 22nd to 24th aspects, wherein while the first ultrasound image is being displayed on the screen, the processor outputs third size information indicating a third size of the range selected according to a given fourth instruction among a plurality of ranges.

A 26th aspect according to the technology of the present disclosure is the medical assistance apparatus according to the 25th aspect, wherein the ranges are assigned a priority, and calipers indicating the ranges are displayed in a state allowing for identification of the priority.

A 27th aspect according to the technology of the present disclosure is the medical assistance apparatus according to the 26th aspect, wherein the calipers are displayed on the screen in an order corresponding to the priority, and the calipers to be displayed on the screen are switched according to a given fifth instruction.

A 28th aspect according to the technology of the present disclosure is the medical assistance apparatus according to any one of the first to 27th aspects, wherein the first size information and/or related information that is related to the first size information are saved in an external apparatus and/or medical record.

A 29th aspect according to the technology of the present disclosure is the medical assistance apparatus according to any one of the first to 28th aspects, wherein the ultrasound image is an endoscopic ultrasound image.

A 30th aspect according to the technology of the present disclosure is a medical assistance apparatus including a processor configured to: detect a target site of observation by performing image recognition processing on an ultrasound image; and measure the target site of observation according to a measurement method appropriate for the detected target site of observation.

A 31st aspect according to the technology of the present disclosure is the medical assistance apparatus according to the 30th aspect, wherein the measurement method includes a first measurement method and/or a second measurement method, the first measurement method is a method for measuring the target site of observation in one direction, and the second measurement method is a method for measuring the target site of observation in multiple directions.

A 32nd aspect according to the technology of the present disclosure is an ultrasound endoscope including: the medical assistance apparatus according to any one of the first to 31st aspects; and an ultrasound probe that, when inserted into a body, emits an ultrasonic wave inside the body and receives a reflected wave of the ultrasonic wave, wherein the ultrasound image is generated on a basis of the reflected wave.

A 33rd aspect according to the technology of the present disclosure is a medical assistance method including: displaying, on a screen, a first ultrasound image among a plurality of ultrasound images in a time series, the ultrasound image showing a target site of observation and satisfying a predetermined condition; and outputting first size information indicating a first size, wherein the first size is a size of the target site of observation shown in the first ultrasound image.

A 34th aspect according to the technology of the present disclosure is a program causing a computer to execute a process including: displaying, on a screen, a first ultrasound image among a plurality of ultrasound images in a time series, the ultrasound image showing a target site of observation and satisfying a predetermined condition; and outputting first size information indicating a first size, wherein the first size is a size of the target site of observation shown in the first ultrasound image.

### BRIEF DESCRIPTION OF THE DRAWINGS

Exemplary embodiments according to the technique of the present disclosure will be described in detail based on the following figures, wherein:

FIG. 1 is a conceptual diagram illustrating an example of an aspect in which an endoscope system is used;

FIG. 2 is a conceptual diagram illustrating an example of an overall configuration of an endoscope system;

FIG. 3 is a block diagram illustrating an example of a configuration of an ultrasound endoscope;

FIG. 4 is a conceptual diagram illustrating an example of details of processing by a generation unit;

FIG. 5 is a conceptual diagram illustrating an example of details of processing by a generation unit, a detection unit, and a measurement unit;

FIG. 6 is a conceptual diagram illustrating an example of details of processing by a generation unit and a control unit;

FIG. 7 is a conceptual diagram illustrating an example of details of processing by a generation unit and a measurement unit;

FIG. 8 is a conceptual diagram illustrating an example of details of processing by a generation unit, a measurement unit, and a control unit;

FIG. 9A is a flowchart illustrating an example of the flow of medical assistance processing;

FIG. 9B is a continuation of the flowchart illustrated in FIG. 9A;

FIG. 10 is a conceptual diagram illustrating an example of an aspect in which a first size and second sizes are displayed on a screen in a distinguishable manner;

FIG. 11 is a conceptual diagram illustrating an example of an aspect in which a size is remeasured in association with a change in the geometric properties of calipers, and the remeasured size is displayed on a screen;

FIG. 12 is a conceptual diagram illustrating an example modification of details of processing by a generation unit, a detection unit, and a measurement unit;

FIG. 13 is a conceptual diagram illustrating an aspect in which the display of calipers is switched according to a switch instruction;

FIG. 14 is a conceptual diagram illustrating an example of an aspect in which the size of a range selected according to a select instruction is displayed on a screen;

FIG. 15 is a conceptual diagram illustrating an example of an aspect in which the major-axis length and the minor-axis length of a cross section of an internal organ shown in an ultrasound image are measured and displayed on a screen;

FIG. 16 is a conceptual diagram illustrating an example of an aspect in which the size of the radial range of a transverse sectional view of a vessel shown in an ultrasound image is measured;

FIG. 17 is a conceptual diagram illustrating an example of an aspect in which the size of the radial range of a longitudinal sectional view of a vessel shown in an ultrasound image is measured;

FIG. 18 is a conceptual diagram illustrating an example of an aspect in which the size of the range from one end to the other end of a line segment traversing the outline of a cross section of an annular lesion shown in an ultrasound image is measured;

FIG. 19 is a conceptual diagram illustrating an example of an aspect in which a processing result of medical assistance processing is outputted to an external apparatus and a printer;

FIG. 20 is a conceptual diagram illustrating an example of an aspect in which a function-enhancing apparatus is connected to a processing apparatus; and

FIG. 21 is a conceptual diagram illustrating an aspect in which the display of calipers with respect to a single area under observation is switched according to a switch instruction.

### DETAILED DESCRIPTION

The following describes, in accordance with the attached drawings, examples of embodiments of a medical assistance apparatus, an ultrasound endoscope, a medical assistance method, and a program according to the technology of the present disclosure.

First, terms used in the following description will be explained.

CPU is an abbreviation for “central processing unit”. GPU is an abbreviation for “graphics processing unit”. TPU is an abbreviation for “tensor processing unit”. RAM is an abbreviation for “random access memory”. NVM is an abbreviation for “non-volatile memory”. EEPROM is an abbreviation for “electrically erasable programmable read-only memory”. ASIC is an abbreviation for “application-specific integrated circuit”. PLD is an abbreviation for “programmable logic device”. FPGA is an abbreviation for “field-programmable gate array”. SoC is an abbreviation for “system-on-a-chip”. SSD is an abbreviation for “solid-state drive”. USB is an abbreviation for “Universal Serial Bus”. HDD is an abbreviation for “hard disk drive”. EL is an abbreviation for “electroluminescence”. CMOS is an abbreviation for “complementary metal-oxide-semiconductor”. CCD is an abbreviation for “charge-coupled device”. WAN is an abbreviation for “wide area network”. AI is an abbreviation for “artificial intelligence”. BLI is an abbreviation for “blue light imaging”. LCI is an abbreviation for “linked color imaging”. NN is an abbreviation for “neural network”.

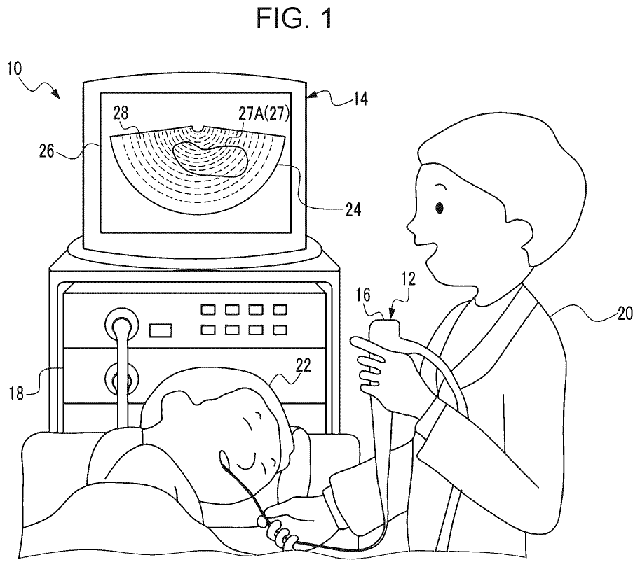

As illustrated by way of example in FIG. 1, an endoscope system 10 is provided with an ultrasound endoscope 12 and a display apparatus 14. The ultrasound endoscope 12 is an ultrasound endoscope of the convex type, and is provided with an ultrasound endoscope main body 16 and a processing apparatus 18. Although the present embodiment gives an ultrasound endoscope of the convex type as an example of the ultrasound endoscope 12, this is merely one example, and the technology of the present disclosure is also achieved with an ultrasound endoscope of the radial type. The ultrasound endoscope 12 is an example of an “ultrasound endoscope” according to the technology of the present disclosure. The processing apparatus 18 is an example of a “medical assistance apparatus” according to the technology of the present disclosure.

The ultrasound endoscope main body 16 is used by a physician 20, for example. The processing apparatus 18 is connected to the ultrasound endoscope main body 16 and exchanges various signals with the ultrasound endoscope main body 16. That is, the processing apparatus 18 may output a signal to the ultrasound endoscope main body 16 to control operations by the ultrasound endoscope main body 16, and perform various types of signal processing on a signal inputted from the ultrasound endoscope main body 16.

The ultrasound endoscope 12 is an apparatus for enabling the physician 20 to observe a target site of observation 27 inside the body of a subject 22 and for carrying out diagnosis and/or treatment of the target site of observation 27, and generates and outputs an ultrasound image 24 indicating an area 28 that includes the target site of observation 27.

In the example illustrated in FIG. 1, an internal organ 27A is illustrated as an example of the target site of observation 27 shown in the ultrasound image 24. The internal organ 27A may be an organ such as the gallbladder or the pancreas, for example. The internal organ 27A is given as an example to facilitate comprehension of the technology of the present disclosure, but this is merely one example, but the target site of observation 27 may also be a massive lesion (for example, a unilocular cyst, a tumor, or a mass) or a vessel (for example, a blood vessel, a lymphatic vessel, a bile duct, a pancreatic duct, or a medical artificial duct).

The target site of observation 27 is an example of a “target site of observation” according to the technology of the present disclosure. The internal organ 27A is an example of an “internal organ” and a “specific target site of observation” according to the technology of the present disclosure. A massive lesion as the target site of observation 27 is an example of a “massive lesion” according to the technology of the present disclosure. A vessel as the target site of observation 27 is an example of a “vessel” according to the technology of the present disclosure.

For example, in the case of observing the area 28 inside the body of the subject 22, the physician 20 inserts the ultrasound endoscope main body 16 into the body of the subject 22 from the mouth or nose (in the example illustrated in FIG. 1, the mouth), and emits an ultrasonic wave at the position of the stomach, duodenum, or the like. The ultrasound endoscope main body 16 emits an ultrasonic wave toward the area 28 inside the body of the subject 22 and detects a reflected wave obtained when the emitted ultrasonic wave is reflected by the area 28.

Note that although the example in FIG. 1 illustrates a situation in which upper gastrointestinal endoscopy is performed, the technology of the present disclosure is not limited thereto, and the technology of the present disclosure is also applicable to lower gastrointestinal endoscopy, bronchoscopy, or the like.

The processing apparatus 18 generates the ultrasound image 24 on the basis of the reflected wave detected by the ultrasound endoscope main body 16, and outputs the generated ultrasound image 24 to the display apparatus 14 or the like.

The display apparatus 14 displays various information, including images, under control by the processing apparatus 18. The display apparatus 14 may be a liquid crystal display or an EL display, for example. The ultrasound image 24 generated by the processing apparatus 18 is displayed as a dynamic image according to a predetermined frame rate (a few dozen frames per second, for example) on a screen 26 of the display apparatus 14. The dynamic image may be a live-view image or a post-view image, for example. The screen 26 is an example of a “screen” according to the technology of the present disclosure.

Note that although the example in FIG. 1 illustrates an example arrangement in which the ultrasound image 24 is displayed on the screen 26 of the display apparatus 14, this is merely one example, and the ultrasound image 24 may also be displayed on a screen of a display apparatus (for example, the display of a tablet terminal) other than the display apparatus 14. The ultrasound image 24 may also be stored in a non-transitory computer readable medium (for example, flash memory, an HDD, and/or magnetic tape).

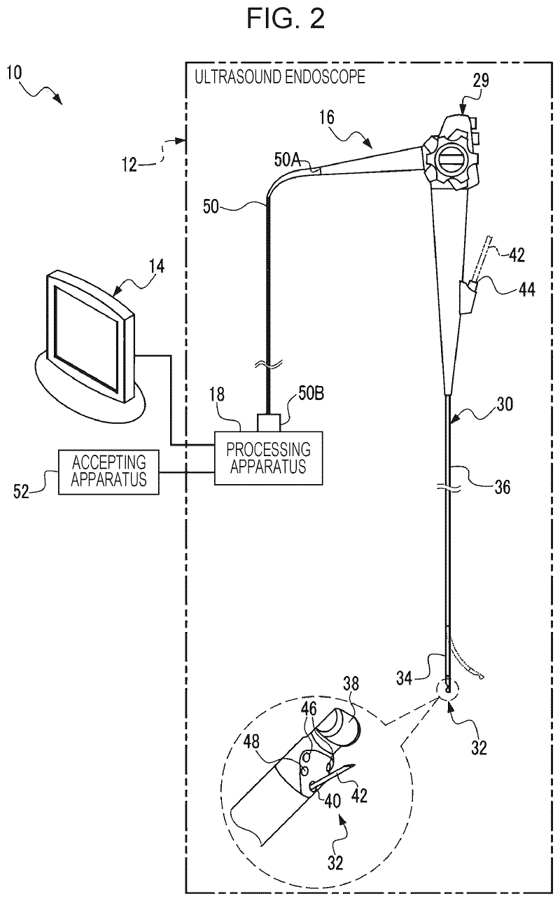

As illustrated by way of example in FIG. 2, the ultrasound endoscope main body 16 is provided with a manipulation part 29 and an insertion part 30. The insertion part 30 is formed into a tubular shape. The insertion part 30 has a leading end part 32, a curving part 34, and a flexible part 36. The leading end part 32, the curving part 34, and the flexible part 36 are disposed from the leading-end side to the base-end side of the insertion part 30 in the order of the leading end part 32, the curving part 34, and the flexible part 36. The flexible part 36 is formed from a long, flexible material, and connects the manipulation part 29 with the curving part 34. Manipulating the manipulation part 29 causes the curving part 34 to partially curve or rotate about the axis of the insertion part 30. As a result, the insertion part 30 is fed deeper into a luminal organ while curving or rotating about the axis of the insertion part 30 according to the shape of a luminal organ (the shape of the duodenum tract, for example).

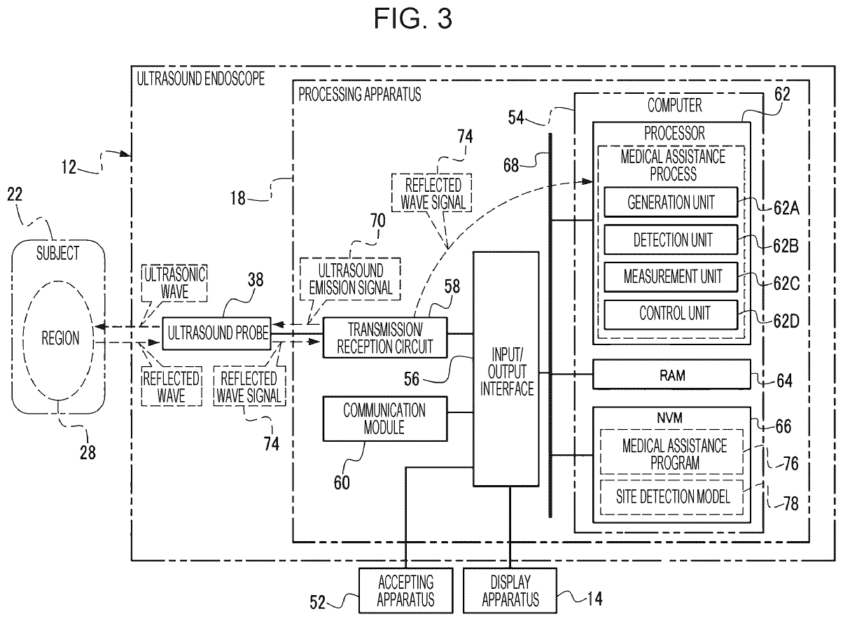

An ultrasound probe 38 and a treatment aperture 40 are provided in the leading end part 32. The ultrasound probe 38 is provided on the leading-end side of the leading end part 32. The ultrasound probe 38 is an ultrasound probe of the convex type that emits an ultrasonic wave and receives a reflected wave obtained when the emitted ultrasonic wave is reflected by the area 28 (see FIGS. 1 and 3).

The treatment aperture 40 is formed closer to the base-end side of the leading end part 32 than the ultrasound probe 38. The treatment aperture 40 is an aperture for allowing a treatment tool 42 to protrude from the leading end part 32. A treatment tool insertion port 44 is formed in the manipulation part 29, and the treatment tool 42 is inserted into the insertion part 30 from the treatment tool insertion port 44. The treatment tool 42 passes through the interior of the insertion part 30 to protrude out of the ultrasound endoscope main body 16 from the treatment aperture 40. The treatment aperture 40 also functions as an aspiration port to aspirate blood, internal contaminants, and the like.

In the example illustrated in FIG. 2, a puncture needle is illustrated as the treatment tool 42. Note that this is merely one example, and the treatment tool 42 may also be grasping forceps, a cannula, a wire, and/or a sheath.

In the example illustrated in FIG. 2, an illumination apparatus 46 and a camera 48 are provided in the leading end part 32. The illumination apparatus 46 radiates light. The type of light radiated from the illumination apparatus 46 may be visible light (white light, for example), non-visible light (near-infrared light, for example), and/or special light, for example. The special light may be light for BLI and/or light for LCI, for example.

The camera 48 images the inside of a luminal organ using an optical method. The camera 48 may be a CMOS camera, for example. A CMOS camera is merely one example, and the camera 48 may also be another type of camera, such as a CCD camera. Note that an image obtained through imaging by the camera 48 is displayed on the display apparatus 14, displayed on a display apparatus (for example, the display of a tablet terminal) other than the display apparatus 14, and/or stored in a storage medium (for example, flash memory, an HDD, and/or magnetic tape).

The ultrasound endoscope 12 is provided with a processing apparatus 18 and a universal cord 50. The universal cord 50 has a base end part 50A and a leading end part 50B. The base end part 50A is connected to the manipulation part 29. The leading end part 50B is connected to the processing apparatus 18. That is, the ultrasound endoscope main body 16 and the processing apparatus 18 are connected via the universal cord 50.

The endoscope system 10 is provided with an accepting apparatus 52. The accepting apparatus 52 is connected to the processing apparatus 18. The accepting apparatus 52 accepts instructions from a user. Examples of the accepting apparatus 52 include: an operation panel with multiple hardware keys and/or a touch panel; a keyboard; a mouse; a trackball; a footswitch; a smart device; a microphone; and/or remote-control equipment.

The processing apparatus 18 performs various types of signal processing and exchanges various signals with the ultrasound endoscope main body 16, according to instructions accepted by the accepting apparatus 52. For example, according to an instruction accepted by the accepting apparatus 52, the processing apparatus 18 causes the ultrasound probe 38 to emit an ultrasonic wave, and generates and outputs the ultrasound image 24 (see FIG. 1) on the basis of a reflected wave received by the ultrasound probe 38.

The display apparatus 14 is connected to the processing apparatus 18. The processing apparatus 18 controls the display apparatus 14 according to instructions accepted by the accepting apparatus 52. This causes, for example, the ultrasound image 24 generated by the processing apparatus 18 to be displayed on the screen 26 of the display apparatus 14 (see FIG. 1).

As illustrated by way of example in FIG. 3, the processing apparatus 18 is provided with a computer 54, an input/output interface 56, a transmission/reception circuit 58, and a communication module 60. The computer 54 is an example of a “computer” according to the technology of the present disclosure.

The computer 54 is provided with a processor 62, RAM 64, and NVM 66. The input/output interface 56, processor 62, RAM 64, and NVM 66 are connected to a bus 68.

The processor 62 controls the processing apparatus 18 overall. For example, the processor 62 includes a CPU and a GPU, and the GPU operates under control by the CPU and is mainly responsible for executing image processing. Note that the processor 62 may also be one or more CPUs with integrated GPU functionality, or one or more CPUs without integrated GPU functionality. The processor 62 may also include a multi-core CPU, and may also include a TPU. The processor 62 is an example of a “processor” according to the technology of the present disclosure.

The RAM 64 is a memory in which information is stored temporarily, and is used as work memory by the processor 62. The NVM 66 is a non-volatile storage apparatus storing various programs, various parameters, and the like. The NVM 66 may be flash memory (EEPROM, for example) and/or an SSD, for example. Note that flash memory and an SSD are merely one example, and the NVM 66 may also be another type of non-volatile storage apparatus, such as an HDD, and may also be a combination of two or more types of non-volatile storage apparatuses.

The accepting apparatus 52 is connected to the input/output interface 56, and the processor 62 acquires an instruction accepted by the accepting apparatus 52 via the input/output interface 56 and executes processing according to the acquired instruction.

The transmission/reception circuit 58 is connected to the input/output interface 56. The transmission/reception circuit 58 generates an ultrasound emission signal 70 with a pulse waveform according to an instruction from the processor 62, and outputs the generated ultrasound emission signal 70 to the ultrasound probe 38. The ultrasound probe 38 converts the ultrasound emission signal 70 inputted from the transmission/reception circuit 58 into an ultrasonic wave and emits the ultrasonic wave toward the area 28 of the subject 22. The ultrasound probe 38 receives a reflected wave obtained when the ultrasonic wave emitted from the ultrasound probe 38 is reflected by the area 28, converts the reflected wave into a reflected wave signal 74, which is an electrical signal, and outputs the reflected wave signal 74 to the transmission/reception circuit 58. The transmission/reception circuit 58 digitizes the reflected wave signal 74 inputted from the ultrasound probe 38 and outputs the digitized reflected wave signal 74 to the processor 62 via the input/output interface 56. The processor 62 generates the ultrasound image 24 (see FIG. 1) illustrating the state of the area 28 (that is, the state of a cross section of the area 28) on the basis of the reflected wave signal 74 inputted from the transmission/reception circuit 58 via the input/output interface 56.

Although omitted from illustration in FIG. 3, the illumination apparatus 46 (see FIG. 2) is also connected to the input/output interface 56. The processor 62 controls the illumination apparatus 46 via the input/output interface 56 to change the type of light radiated from the illumination apparatus 46 and to adjust the light intensity. Although omitted from illustration in FIG. 3, the camera 48 (see FIG. 2) is also connected to the input/output interface 56. The processor 62 controls the camera 48 via the input/output interface 56 and acquires, via the input/output interface 56, an image obtained by having the camera 48 image the inside of the body of the subject 22.

The communication module 60 is connected to the input/output interface 56. The communication module 60 is an interface including a communication processor, an antenna, and the like. The communication module 60 is connected to a LAN, WAN, or other network (not illustrated), and directs communication between the processor 62 and an external apparatus. The external apparatus may be a server (for example, an electronic medical record management server and/or image management server), a tablet terminal, and/or a personal computer, for example.

The display apparatus 14 is connected to the input/output interface 56, and the processor 62 controls the display apparatus 14 via the input/output interface 56, thereby causing the display apparatus 14 to display various information.

The accepting apparatus 52 is connected to the input/output interface 56, and the processor 62 acquires an instruction accepted by the accepting apparatus 52 via the input/output interface 56 and executes processing according to the acquired instruction.

Incidentally, it is important for the physician 20 observing the ultrasound image 24 displayed on the screen 26 to ascertain the size of the target site of observation 27 in order to perform some kind of medical treatment on the target site of observation 27 and/or to evaluate the progress of the target site of observation 27, for example. The size of the target site of observation 27 shown in the ultrasound image 24 changes depending on how the target site of observation 27 is shown in the ultrasound image 24.

Consequently, for example, if the target site of observation 27 is shown in the ultrasound image 24 at a minimum size, and the size of the target site of observation 27 is measured according to some method and displayed on the screen 26, it is highly probable that the size displayed on the screen 26 will not be the size that the physician 20 actually wants to know. Moreover, even if the ultrasound image 24 is selected according to an instruction by the physician 20 among a plurality of ultrasound images 24 in a time series being displayed as a dynamic image on the screen 26, and the size of the target site of observation 27 shown in the selected ultrasound image 24 is measured and displayed on the screen 26, it is not necessarily the case that the size of the target site of observation 27 that is desired by the physician 20 will be displayed on the screen 26.

As an example, the physician 20 may intend to select the ultrasound image 24 that shows the target site of observation 27 at its largest, but because the dynamic image is being displayed at a high speed, the ultrasound image 24 that is temporally ahead of or behind the ultrasound image 24 showing the target site of observation 27 at its largest may be selected accidentally. As a result, a size of the target site of observation 27 that is not desired by the physician 20 is displayed on the screen 26.

In light of such circumstances, in the present embodiment, medical assistance processing is performed by the processor 62 of the processing apparatus 18, as illustrated by way of example in FIG. 3. The medical assistance processing detects the target site of observation 27 from the area 28 according to an AI approach, and uses a detection result as a basis for assisting with medical care (for example, medical care performed by the physician 20 (see FIG. 1)) using the ultrasound endoscope 12.

A medical assistance program 76 and a site detection model 78 are stored in the NVM 66. The medical assistance program 76 is an example of a “program” according to the technology of the present disclosure. The site detection model 78 is used in the processing by which the processor 62 detects the target site of observation 27 from the ultrasound image 24.

The processor 62 performs the medical assistance processing by reading out the medical assistance program 76 from the NVM 66 and executing the read medical assistance program 76 in the RAM 64. The detection of the target site of observation 27 according to an AI approach is achieved through the use of the site detection model 78. The medical assistance processing is achieved by the processor 62 operating as a generation unit 62A, a detection unit 62B, a measurement unit 62C, and a control unit 62D according to the medical assistance program 76 executed in the RAM 64.

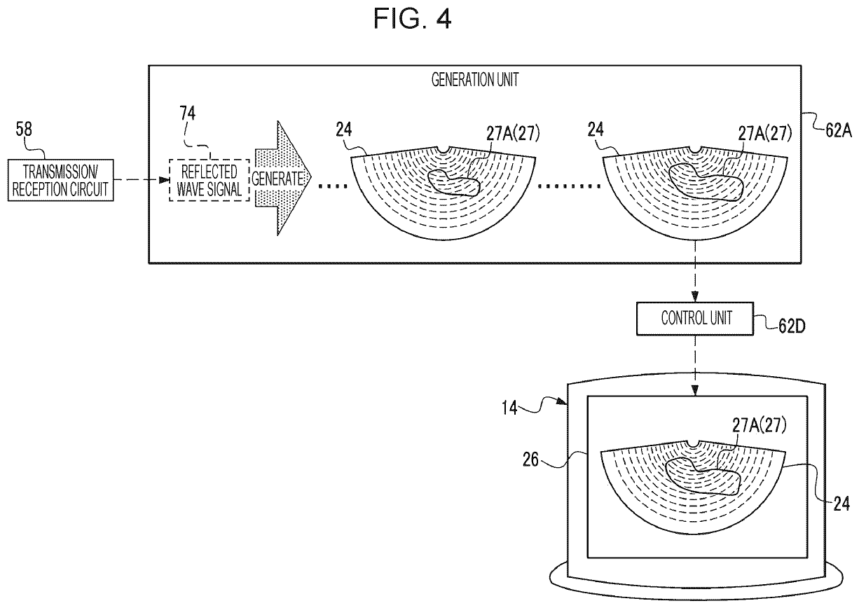

As illustrated by way of example in FIG. 4, the generation unit 62A acquires the reflected wave signal 74 from the transmission/reception circuit 58 and generates a plurality of ultrasound images 24 in a time series on the basis of the acquired reflected wave signal 74. Note that in the present embodiment, for convenience, the description assumes that the same target site of observation 27 is shown in the plurality of ultrasound images 24 in a time series. Also, in the present embodiment, the plurality of ultrasound images 24 in a time series generated by the generation unit 62A are an example of “a plurality of ultrasound images in a time series” according to the technology of the present disclosure. The ultrasound image 24 is an example of an “image generated by an ultrasound method” and an “endoscopic ultrasound image” according to the technology of the present disclosure.

The control unit 62D displays the plurality of ultrasound images 24 in a time series generated by the generation unit 62A on the screen 26 as a dynamic image at a specified frame rate (for example, a few dozen frames per second).

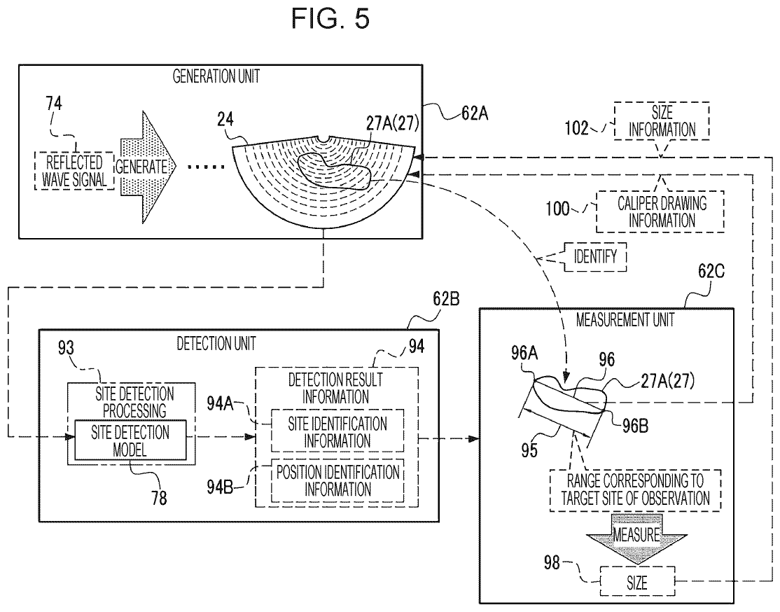

As illustrated by way of example in FIG. 5, the detection unit 62B acquires the ultrasound image 24 one frame at a time following the time series from the plurality of ultrasound images 24 in a time series generated by the generation unit 62A, and executes site detection processing 93 on the acquired ultrasound image 24. The site detection processing 93 detects, according to the site detection model 78, the target site of observation 27 shown in the ultrasound image 24. In the present embodiment, the site detection processing 93 is an example of “image recognition processing” according to the technology of the present disclosure.

The site detection model 78 is a trained model for object detection according to an AI approach, and has been optimized by causing a neural network to undergo machine learning using first labeled training data. The first labeled training data is a plurality of data (that is, data of multiple frames) in which first example data and first ground truth data are associated with one another.

The first example data contains images corresponding to the ultrasound image 24. The first ground truth data contains ground truth data (that is, annotations) with respect to the first example data. As an example of the first ground truth data, annotations that can be used to identify sites corresponding to the target site of observation 27 in images corresponding to the ultrasound image 24 (for example, information indicating the name of the target site of observation 27, a plurality of coordinates that can be used to identify the position of a site corresponding to the target site of observation 27, and/or the like) are used.

The detection unit 62B inputs the ultrasound image 24 acquired from the generation unit 62A into the site detection model 78. This causes the site detection model 78 to detect the target site of observation 27 shown in the inputted ultrasound image 24 and output detection result information 94 indicating a detection result. The detection unit 62B acquires the detection result information 94 outputted from the site detection model 78.

The detection result information 94 includes site identification information 94A and position identification information 94B. The detection result information 94 is information indicating the name of the target site of observation 27 shown in the ultrasound image 24. In the example illustrated in FIG. 5, the name of the target site of observation 27 refers to the name (for example, pancreas) of the internal organ 27A shown in the ultrasound image 24, for example. The position identification information 94B is position information (for example, a plurality of coordinates) that can be used to identify the location where the target site of observation 27 is shown within the ultrasound image 24.

Note that in the present embodiment, processing involving an AI approach using the site detection model 78 is given as an example of the site detection processing 93, but the technology of the present disclosure is not limited thereto, and the site detection processing 93 can also be achieved through joint use of processing involving an AI approach and processing involving a non-AI approach (for example, processing using template matching or the like), or processing involving a non-AI approach instead of processing involving an AI approach.

The measurement unit 62C refers to the position identification information 94B to identify the target site of observation 27 from the ultrasound image 24 to be processed by the site detection processing 93. The measurement unit 62C refers to the site identification information 94A to identify a range 95 corresponding to the target site of observation 27 identified from the ultrasound image 24. The range 95 is defined for each target site of observation 27. For example, the range 95 to be applied to the internal organ 27A is the range within which organ length reaches a maximum in cross sections of the internal organ 27A shown in the ultrasound images 24.

Note that although the description herein gives the range within which organ length reaches a maximum in cross sections of the internal organ 27A as an example of the range 95 to be applied to the internal organ 27A, this is merely one example, and even if the target site of observation 27 is a massive lesion, the range within which lesion length reaches a maximum in cross sections of the massive lesion shown in the ultrasound images 24 is applied as the range 95. Also, although described in detail later, in the case where the target site of observation 27 is a vessel (for example, a bile duct or a pancreatic duct), a range in the radial direction of the vessel (for example, the diameter) is applied as the range 95.

The measurement unit 62C generates calipers 96 that define the range 95. The calipers 96 are an image (for example, linear marks) enabling visual recognition of where the range 95 begins and ends. The interval from one end 96A to the other end 96B of the calipers 96 represents the range 95.

The measurement unit 62C generates caliper drawing information 100, which is information that can be used to draw the calipers 96 within the ultrasound image 24, and adds the generated caliper drawing information 100 to the ultrasound image 24 showing the target site of observation 27 to which the range 95 is applied. In other words, the measurement unit 62C adds the caliper drawing information 100 to each of the plurality of ultrasound images 24 to be processed by the site detection processing 93.

In one example, the caliper drawing information 100 may be a plurality of coordinates that can be used to identify the position of the calipers 96 within the ultrasound image 24 (for example, coordinates that can be used to identify the one end 96A and coordinates that can be used to identify the other end 96B within the ultrasound image 24). The addition of the caliper drawing information 100 to the ultrasound image 24 is achieved by, for example, storing the caliper drawing information 100 and an identifier that can be used to identify the ultrasound image 24 in association with each other in a memory (for example, the RAM 64 and/or the NVM 66).

The measurement unit 62C measures the size 98 (as an example herein, the length) of the range 95. The measurement unit 62C then adds size information 102 indicating the measured size 98 to the ultrasound image 24 to be processed by the site detection processing 93 (that is, the ultrasound image 24 showing the target site of observation 27 to which the range 95 is applied). In other words, the measurement unit 62C adds the size information 102 to each of the plurality of ultrasound images 24 to be processed by the site detection processing 93. The addition of the size information 102 to the ultrasound image 24 is achieved by, for example, storing the size information 102 and an identifier that can be used to identify the ultrasound image 24 in association with each other in a memory.

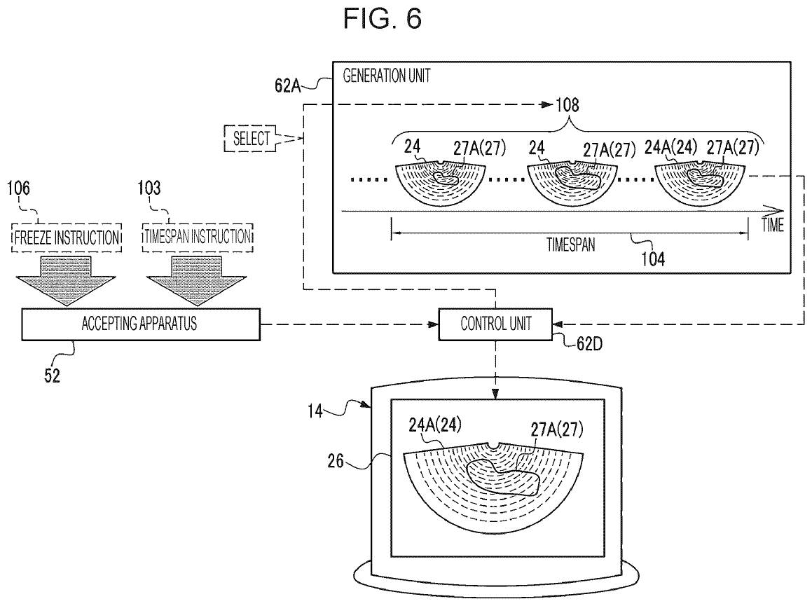

As illustrated by way of example in FIG. 6, in a situation in which the plurality of ultrasound images 24 in a time series are displayed as a dynamic image on the screen 26, the control unit 62D freezes (that is, pauses) the ultrasound image 24 on the screen 26 according to a freeze instruction 103 given to the endoscope system 10. The freeze instruction 103 is an instruction for freezing the display of the ultrasound image 24. In the example illustrated in FIG. 6, in a situation in which the plurality of ultrasound images 24 in a time series are being displayed as a dynamic image on the screen 26, the control unit 62D freezes the ultrasound image 24 on the screen 26 when the freeze instruction 103 is accepted by the accepting apparatus 52.

Using a freeze image 24A as a reference point, the control unit 62D sets a timespan 104 according to a timespan instruction 106 given to the endoscope system 10 (in the example illustrated in FIG. 6, a timespan instruction 106 accepted by the accepting apparatus 52). The freeze image 24A refers to the ultrasound image 24 displayed in a frozen state on the screen 26. The timespan instruction 106 refers to an instruction regarding the length of the timespan 104. In the example illustrated in FIG. 6, the control unit 62D sets, as the timespan 104, a timespan going back according to the timespan instruction 106 from the point in time when the freeze image 24A is obtained among the plurality of ultrasound images in a time series generated by the generation unit 62A. The control unit 62D then selects the plurality of ultrasound images 24 in a time series included within the timespan 104 as an image group 108 within the timespan.

In the present embodiment, the freeze instruction 103 is an example of a “first instruction” according to the technology of the present disclosure. The timespan instruction 106 is an example of a “second instruction” according to the technology of the present disclosure. The freeze image 24A is an example of a “selected ultrasound image” and a “freeze image” according to the technology of the present disclosure. The timespan 104 is an example of a “timespan” according to the technology of the present disclosure.

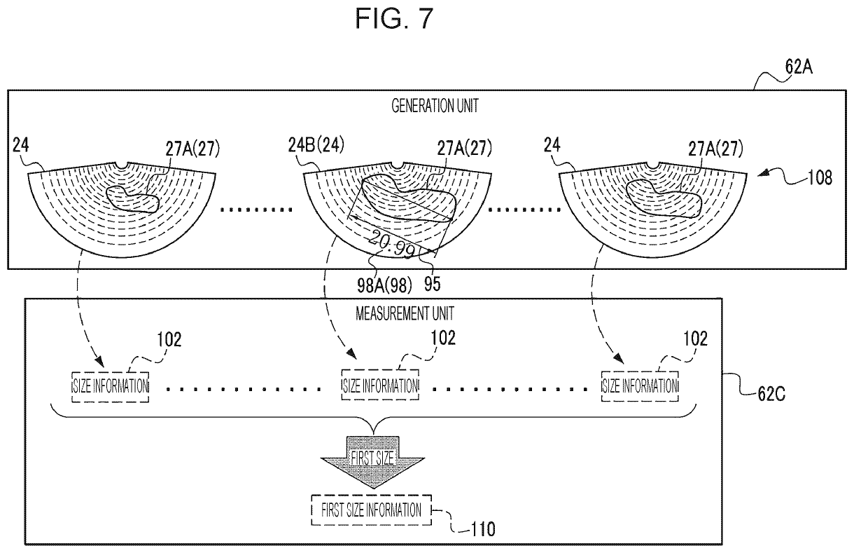

As illustrated by way of example in FIG. 7, the measurement unit 62C measures a first size 98A, which is the size 98 of the target site of observation 27 shown in a first ultrasound image 24B belonging to the image group 108 within the timespan. In other words, the measurement unit 62C acquires a plurality of size information 102 (for example, all of the size information 102) applied to the image group 108 within the timespan, and selects first size information 110 indicating the first size 98A among the acquired plurality of size information 102.

The first size 98A may be the size 98 of the range 95 corresponding to the target site of observation 27 included in the first ultrasound image 24B, for example. The first ultrasound image 24B refers to the ultrasound image 24 that satisfies a predetermined condition from out of the image group 108 within the timespan. The predetermined condition includes a condition stipulating that the image from out of the image group 108 within the timespan is an image in which the target site of observation 27 (as an example herein, the internal organ 27A) is detected by the detection unit 62B and the size 98 of the detected target site of observation 27 is measured by the measurement unit 62C. The predetermined condition also includes a condition stipulating that the size 98 of the range 95 (that is, the length of the range 95) is the maximum value, for example.

The above gives a condition stipulating that the size 98 of the range 95 is the maximum value as an example of the predetermined condition, but this is merely one example, and the predetermined condition may also be, for example, a condition stipulating that the size 98 of the range 95 is the mode or a condition stipulating that the size 98 of the range 95 is equal to or greater than a reference value (for example, a statistical value such as the average or the median of the size 98 of the range 95). The predetermined condition may also stipulate that the size 98 of the range 95 is equal to or greater than a reference value and that the size 98 of the range 95 is the maximum value or the mode. The predetermined condition may also include a condition stipulating that the image quality (for example, the brightness and/or contrast) is at least a predetermined image quality.

In the present embodiment, the first size 98A is an example of a “first size” according to the technology of the present disclosure. The first size information 110 is an example of “first size information” according to the technology of the present disclosure. The first ultrasound image 24B is an example of a “first ultrasound image” according to the technology of the present disclosure.

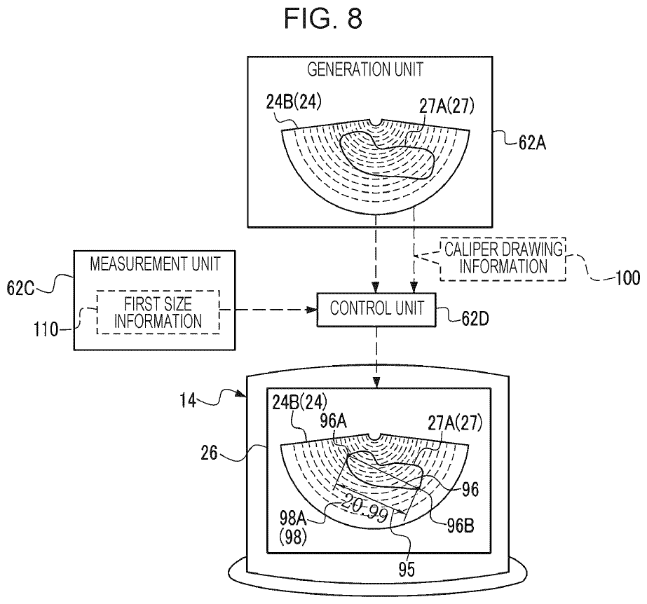

As illustrated by way of example in FIG. 8, the control unit 62D acquires the first ultrasound image 24B from the generation unit 62A and displays the acquired first ultrasound image 24B on the screen 26. The control unit 62D also acquires the caliper drawing information 100 applied to the first ultrasound image 24B. The control unit 62D displays the calipers 96 within the first ultrasound image 24B by drawing, according to the acquired caliper drawing information 100, the calipers 96 defining the range 95 within the first ultrasound image 24B. The control unit 62D also acquires the first size information 110 from the measurement unit 62C and outputs the acquired first size information 110 to the screen 26. That is, the control unit 62D displays on the screen 26 the first size 98A indicated by the first size information 110 acquired from the measurement unit 62C.

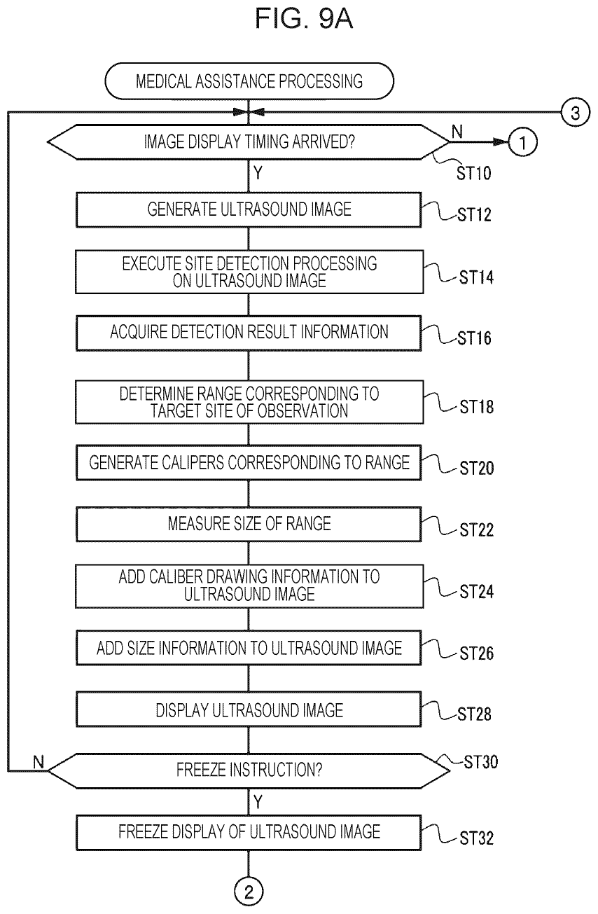

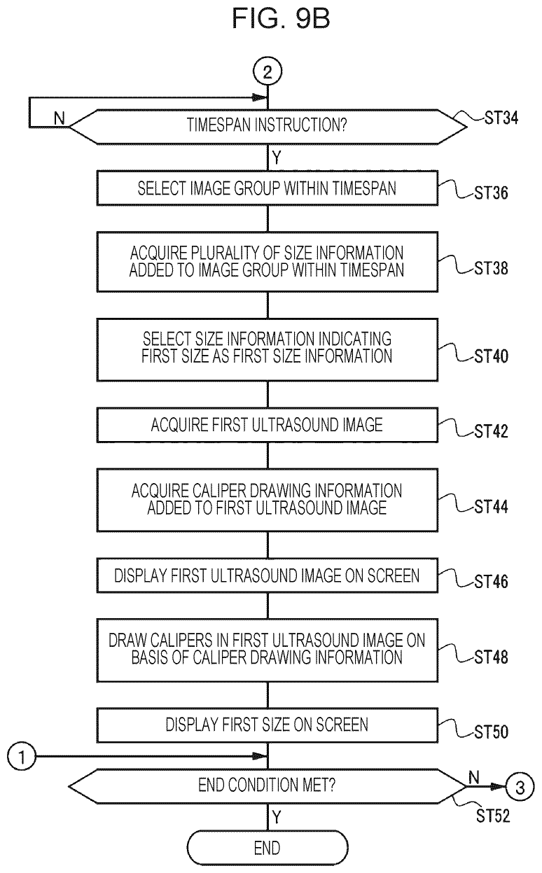

Next, the operation of the portions of the endoscope system 10 according to the technology of the present disclosure will be described with reference to FIGS. 9A and 9B.

FIGS. 9A and 9B are flowcharts illustrating an example of the flow of the medical assistance processing performed by the processor 62. The flow of the medical assistance processing illustrated in FIGS. 9A and 9B is an example of a “medical assistance method” according to the technology of the present disclosure.

In the medical assistance method illustrated in FIG. 9A, first, in step ST10, the generation unit 62A determines whether or not an image display timing has arrived. The image display timings are, for example, timings separated by a time interval defined by the reciprocal of the frame rate. In step ST10, if an image display timing has not arrived, the determination is negative and the medical assistance processing proceeds to step ST52 illustrated in FIG. 9B. In step ST10, if an image display timing has arrived, the determination is positive and the medical assistance processing proceeds to step ST12.

In step ST12, the generation unit 62A generates the ultrasound image 24 on the basis of the reflected wave signal 74 inputted from the transmission/reception circuit 58 (see FIGS. 4 and 5). After the processing in step ST12 is executed, the medical assistance processing proceeds to step ST14.

In step ST14, the detection unit 62B executes the site detection processing 93 (see FIG. 6) using the site detection model 78 on the ultrasound image 24 generated in step ST12. By causing the detection unit 62B to execute the site detection processing 93, the target site of observation 27 shown in the ultrasound image 24 is detected, and the detection result information 94 is outputted from the site detection model 78. After the processing in step ST14 is executed, the medical assistance processing proceeds to step ST16.

In step ST16, the detection unit 62B acquires the detection result information 94 outputted from the site detection model 78 (see FIG. 5). After the processing in step ST16 is executed, the medical assistance processing proceeds to step ST18.

In step ST18, the measurement unit 62C uses the detection result information 94 acquired in step ST16 as a basis for identifying the target site of observation 27 shown in the ultrasound image 24 to be processed by the site detection processing 93. In addition, the measurement unit 62C determines the range 95 corresponding to the identified target site of observation 27 (see FIG. 5).

After the processing in step ST18 is executed, the medical assistance processing proceeds to step ST20.

In step ST20, the measurement unit 62C generates the calipers 96 corresponding to the range 95 determined in step ST18 (see FIG. 5). After the processing in step ST20 is executed, the medical assistance processing proceeds to step ST22.

In step ST22, the measurement unit 62C measures the size 98 of the range 95 determined in step ST18 (see FIG. 5). After the processing in step ST22 is executed, the medical assistance processing proceeds to step ST24.

In step ST24, the measurement unit 62C adds the caliper drawing information 100 pertaining to the calipers 96 generated in step ST20 to the ultrasound image 24 showing the target site of observation 27 to which the range 95 obtained by measuring the size 98 was applied in step ST22 (see FIG. 5). After the processing in step ST24 is executed, the medical assistance processing proceeds to step ST26.

In step ST26, the measurement unit 62C adds the size information 102 indicating the size 98 measured in step ST22 to the ultrasound image 24 showing the target site of observation 27 to which the range 95 obtained by measuring the size 98 was applied in step ST22 (see FIG. 5). After the processing in step ST26 is executed, the medical assistance processing proceeds to step ST28.

In step ST28, the control unit 62D displays the ultrasound image 24 generated in step ST12 on the screen 26 (see FIG. 4). After the processing in step ST28 is executed, the medical assistance processing proceeds to step ST30.

In step ST30, the control unit 62D determines whether or not the accepting apparatus 52 has accepted the freeze instruction 103 (see FIG. 6). In step ST30, if the accepting apparatus 52 has not accepted the freeze instruction 103, the determination is negative and the medical assistance processing proceeds to step ST10. In step ST30, if the accepting apparatus 52 has accepted the freeze instruction 103, the determination is positive and the medical assistance processing proceeds to step ST32.

In step ST32, the control unit 62D freezes the ultrasound image 24 on the screen 26. This causes the freeze image 24A to be displayed on the screen 26 (see FIG. 6). After the processing in step ST32 is executed, the medical assistance processing proceeds to step ST34 illustrated in FIG. 9B.

In step ST34, the control unit 62D determines whether or not the accepting apparatus 52 has accepted the timespan instruction 106 (see FIG. 6). In step ST34, if the accepting apparatus 52 has not accepted the timespan instruction 106, the determination is negative and the determination in step ST34 is made again. In step ST34, if the accepting apparatus 52 has accepted the timespan instruction 106, the determination is positive and the medical assistance processing proceeds to step ST36.

In step ST36, the control unit 62D sets, as the timespan 104, a timespan going back according to the timespan instruction 106 from the point in time when the freeze image 24A is obtained among the plurality of ultrasound images in a time series generated by repeated execution of the processing in step ST12. The control unit 62D then selects the plurality of ultrasound images 24 in a time series included within the timespan 104 as the image group 108 within the timespan (see FIG. 6). After the processing in step ST36 is executed, the medical assistance processing proceeds to step ST38.

In step ST38, the measurement unit 62C acquires the plurality of size information 102 applied to the image group 108 within the timespan selected in step ST36 (see FIG. 7). After the processing in step ST38 is executed, the medical assistance processing proceeds to step ST40.

In step ST40, the measurement unit 62C selects, as the first size information 110, the size information 102 indicating the first size 98A among the plurality of size information 102 acquired in step ST38 (see FIG. 7). After the processing in step ST40 is executed, the medical assistance processing proceeds to step ST42.

In step ST42, the control unit 62D acquires the first ultrasound image 24B, that is, the ultrasound image 24 showing the target site of observation 27 in a manner that satisfies the predetermined condition, from out of the image group within the timespan selected in step ST36 (see FIG. 8). After the processing in step ST42 is executed, the medical assistance processing proceeds to step ST44.

In step ST44, the control unit 62D acquires the caliper drawing information 100 applied to the first ultrasound image 24B (see FIG. 8). After the processing in step ST44 is executed, the medical assistance processing proceeds to step ST46.

In step ST46, the control unit 62D displays the first ultrasound image 24B acquired in step ST42 on the screen 26 (see FIG. 8). After the processing in step ST46 is executed, the medical assistance processing proceeds to step ST48.

In step ST48, the control unit 62D displays the calipers 96 within the first ultrasound image 24B by drawing the calipers 96 defining the range 95 within the first ultrasound image 24B on the screen 26 on the basis of the caliper drawing information 100 acquired in step ST44 (see FIG. 8). After the processing in step ST48 is executed, the medical assistance processing proceeds to step ST50.

In step ST50, the control unit 62D displays on the screen 26 the first size 98A indicated by the first size information 110 selected in step ST40 (see FIG. 8). After the processing in step ST50 is executed, the medical assistance processing proceeds to step ST52.

In step ST52, the control unit 62D determines whether or not a condition for ending the medical assistance processing is met. The condition for ending the medical assistance processing may be, for example, a condition stipulating that an instruction for ending the medical assistance processing has been given to the endoscope system 10 (for example, a condition stipulating that the accepting apparatus 52 has accepted the instruction for ending the medical assistance processing).

In step ST52, if the condition for ending the medical assistance processing is not met, the determination is negative and the medical assistance processing proceeds to step ST10 illustrated in FIG. 9A. In step ST52, if the condition for ending the medical assistance processing is met, the determination is positive and the medical assistance processing ends.

As described above, in the endoscope system 10, the first ultrasound image 24B showing the target site of observation 27 in a manner that satisfies the predetermined condition is displayed on the screen 26 from out of the plurality of ultrasound images 24 in a time series showing the same target site of observation 27. The first size information 110 indicating the first size 98A is outputted. The first size 98A is the size of the target site of observation 27 shown in the first ultrasound image 24B. Consequently, this enables the physician 20 to ascertain the size of the target site of observation 27, the physician 20 observing an ultrasound image 24 (namely the first ultrasound image 24B) which shows the target site of observation 27 in a manner that satisfies the predetermined condition.

In the endoscope system 10, the first size 98A is displayed in correspondence with the first ultrasound image 24B on the screen 26 on which the first ultrasound image 24B is displayed. Consequently, this enables the physician 20 observing the first ultrasound image 24B to visually ascertain the size of the target site of observation 27.

In the endoscope system 10, the size 98 of the range 95 of the target site of observation 27 detected by execution of the site detection processing 93 is measured as the first size 98A, and the first size 98A is displayed on the screen 26. Consequently, this enables the physician 20 to conveniently ascertain the size 98 of the range 95 of the target site of observation 27 intended by the physician 20 as the first size 98A.

In the endoscope system 10, the size information 102 is applied to each ultrasound image 24 included in the image group 108 within the timespan, and the size information 102 applied to the first ultrasound image 24B (that is, the size information 102 applied to the first ultrasound image 24B in the background) is outputted as the first size information 110. Consequently, this enables the physician 20 observing the first ultrasound image 24B to quickly ascertain the first size 98A.

In the endoscope system 10, the first ultrasound image 24B showing the target site of observation 27 in a manner that satisfies the predetermined condition is displayed on the screen 26 from out of the plurality of ultrasound images 24 in a time series showing the same target site of observation 27. A condition stipulating that a length is the maximum value is used as the predetermined condition. Consequently, this enables the physician 20 to ascertain the size 98 that the physician 20 wants to know. Note that the predetermined condition may also be a condition stipulating that a length is equal to or greater than a reference value, and/or a condition stipulating that a length is the mode. These cases likewise enable the physician 20 to ascertain the size 98 that the physician 20 wants to know.

In the endoscope system 10, the ultrasound image 24 showing the target site of observation 27 in a manner that satisfies the predetermined condition is displayed on the screen 26 as the first ultrasound image 24B from out of the plurality of ultrasound images 24 (namely the image group 108 within the timespan) included within the timespan 104 defined on the basis of the freeze image 24A selected among the plurality of ultrasound images 24 according to the freeze instruction 103 accepted by the accepting apparatus 52. Consequently, this enables the physician 20 to ascertain the size 98 of the target site of observation 27 shown in the ultrasound image 24 included in the timespan 104 intended by the physician 20 observing the plurality of ultrasound images 24 as a dynamic image.

In the endoscope system 10, the length of the timespan 104 is defined according to the timespan instruction 106 accepted by the accepting apparatus 52. Consequently, this enables the physician 20 to ascertain the size 98 of the target site of observation 27 shown in the plurality of ultrasound images 24 included within the timespan 104 having the length intended by the physician 20.

In the endoscope system 10, the size 98 of the range 95 corresponding to the target site of observation 27 is measured as the first size 98A and displayed on the screen 26. Consequently, this enables the physician 20 observing the first ultrasound image 24B to ascertain the size 98 of the range 95 corresponding to the target site of observation 27 as the first size 98A.

In the endoscope system 10, the size 98 of the range 95 corresponding to the target site of observation 27 detected by execution of the site detection processing 93 is measured as the first size 98A. Consequently, this allows for measurement of the size 98 of the range 95 corresponding to the target site of observation 27 intended by the physician 20 as the first size 98A.

In the endoscope system 10, in the case where the target site of observation 27 is the internal organ 27A, a cross section of the internal organ 27A is applied as the range 95. Consequently, this enables the physician 20 observing the first ultrasound image 24B showing the internal organ 27A to ascertain the cross-sectional size of the internal organ 27A. Note that in the case where the target site of observation 27 is a massive lesion, a cross section of the massive lesion is similarly applied as the range 95, thus enabling the physician 20 observing the first ultrasound image 24B showing the massive lesion to ascertain the cross-sectional size of the massive lesion.

In the endoscope system 10, the calipers 96 are displayed within the first ultrasound image 24B on the screen 26. The calipers 96 define the range 95. Consequently, this enables the physician 20 observing the first ultrasound image 24B to visually ascertain which range 95 of the target site of observation 27 the size 98 corresponds to when ascertaining the size 98 of the target site of observation 27.

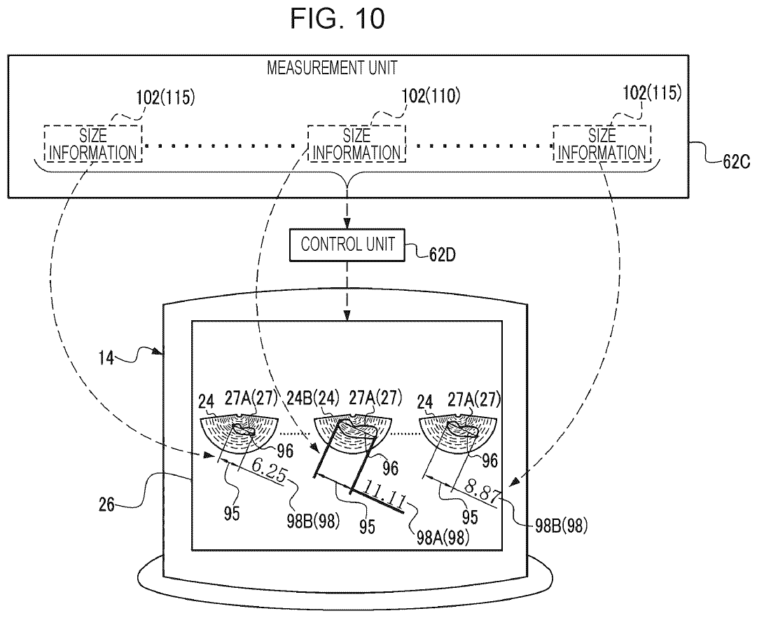

The embodiment above is described using an example in which the control unit 62D outputs the first size information 110 to the display apparatus 14, but the technology of the present disclosure is not limited thereto. For example, as illustrated in FIG. 10, second size information 115, which is size information 102 different from the first size information 110, may also be outputted to the display apparatus 14 in a manner distinguishable from the first size information 110.

The second size information 115 is the size information 102 applied to an ultrasound image 24 different from the first ultrasound image 24B. The second size information 115 is information indicating a second size 98B. The second size 98B is the size 98 of the range 95 of the target site of observation 27 shown in an ultrasound image 24 different from the first ultrasound image 24B. In the example illustrated in FIG. 10, under control by the control unit 62D, a plurality of ultrasound images 24 (for example, the image group 108 within the timespan (see FIG. 6)) are displayed in a list on the screen 26, with the first size 98A and second sizes 98B displayed with a distinguishable display appearance within the respectively corresponding ultrasound images 24.

In the example illustrated in FIG. 10, the second sizes 98B are displayed on the screen 26 in correspondence with the ranges 95 of the ultrasound images 24 different from the first ultrasound image 24B, while the first size 98A is displayed in correspondence with the range 95 within the first ultrasound image 24B and with a more prominent display appearance than the second sizes 98B. In the example illustrated in FIG. 10, the first size 98A is drawn with thicker lines than the second sizes 98B, but this is merely one example. For instance, the first size 98A may also be presented more prominently than the second sizes 98B by altering the brightness and/or adding a border.

In this way, the size 98 (namely the second size 98B) indicated by the size information 102 (namely the second size information 115) applied to an ultrasound image 24 different from the first ultrasound image 24B is displayed on the screen 26, thus enabling the physician 20 to ascertain the size 98 (namely the second size 98B) of the range 95 of the target site of observation 27 shown in an ultrasound image 24 different from the first ultrasound image 24B. Also, since the size information 102 applied to the ultrasound image 24 (namely the size information 102 applied to the ultrasound image 24 in the background) is used as the second size information 115, the second size 98B can be presented to the physician 20 quickly. Also, the first size 98A and the second size 98B are displayed with distinguishable display appearances on the screen 26, thus enabling the physician 20 to distinguishably ascertain the size 98 (namely the first size 98A) of the range 95 of the first ultrasound image 24B and the size 98 (namely the second size 98B) of the range 95 of an ultrasound image 24 different from the first ultrasound image 24B.

Note that in the example illustrated in FIG. 10, the second size 98B is an example of a “second size” according to the technology of the present disclosure. The second size information 115 is an example of “second size information” according to the technology of the present disclosure. The ultrasound image 24 different from the first ultrasound image 24B is an example of a “second ultrasound image” according to the technology of the present disclosure.

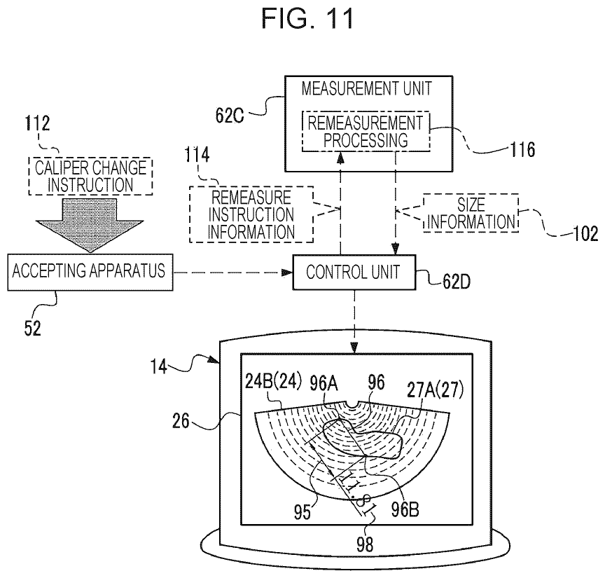

The embodiment above gives an example of a fixed range 95, but the technology of the present disclosure is not limited thereto, and the range 95 may also be changed according to an instruction given by the physician 20. In this case, as illustrated by way of example in FIG. 11, the accepting apparatus 52 may accept a caliper change instruction 112 while the first ultrasound image 24B, the calipers 96, and the first size 98A are being displayed on the screen 26. The caliper change instruction 112 is an instruction causing the control unit 62D to change the geometric properties (for example, the position and length) of the calipers 96. In the example illustrated in FIG. 11, the caliper change instruction 112 is an example of a “third instruction” according to the technology of the present disclosure.

The control unit 62D changes the range 95 by changing the geometric properties of the calipers 96 according to the caliper change instruction 112 accepted by the accepting apparatus 52. Accordingly, the control unit 62D outputs remeasure instruction information 114 to the measurement unit 62C. The remeasure instruction information 114 causes the measurement unit 62C to remeasure the changed range 95. The remeasure instruction information 114 includes information that can be used to identify the geometric properties of the changed calipers 96 (for example, information (such as coordinates) that can be used to identify the position of the one end 96A within the first ultrasound image 24B and information (such as coordinates) that can be used to identify the position of the other end 96B within the first ultrasound image 24B).

The measurement unit 62C performs remeasurement processing 116 according to the inputted remeasure instruction information 114. The remeasurement processing 116 remeasures the size 98 of the range 95 specified according to the geometric properties of the changed calipers 96. The measurement unit 62C outputs, to the control unit 62D, size information 102 indicating the remeasured size 98 obtained by performing the remeasurement processing 116. The control unit 62D displays the size 98 indicated by the inputted size information 102 on the screen 26, in correspondence with the calipers 96.

This enables the physician 20 to ascertain the size 98 of the range 95 intended by the physician 20 when enabling the physician 20 observing the first ultrasound image 24B to ascertain the size 98 of the range 95 of the target site of observation 27.

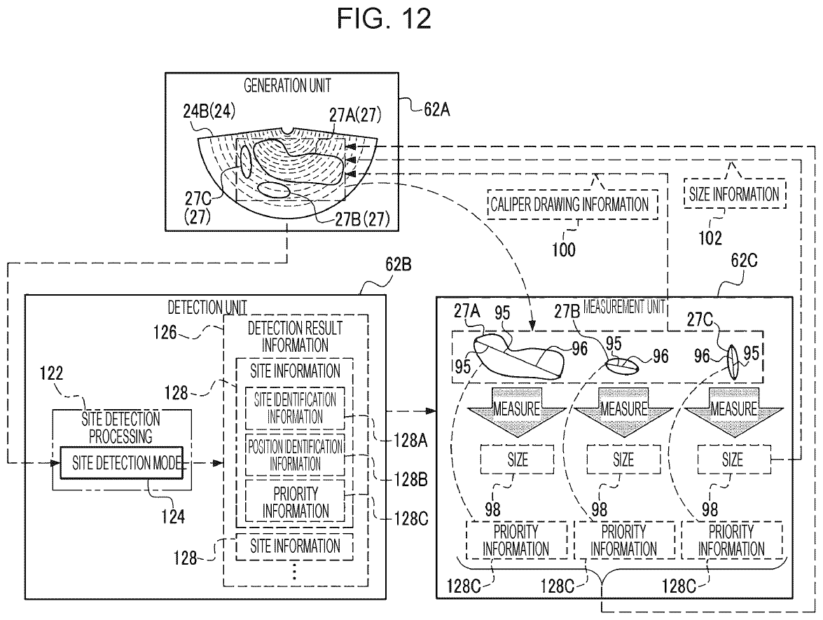

The embodiment above gives an example in which the size 98 (namely the first size 98A) of one range 95 applied to the first ultrasound image 24B is displayed on the screen 26, but the technology of the present disclosure is not limited thereto. For example, as illustrated in FIGS. 12 to 14, a plurality of ranges 95 may be identified from the first ultrasound image 24B, and size information 102 indicating the size 98 of a range 95 selected according to an instruction from the physician 20 may be outputted to the display apparatus 14.

In this case, first, as illustrated by way of example in FIG. 12, the detection unit 62B acquires the first ultrasound image 24B and executes site detection processing 122 on the first ultrasound image 24B. The site detection processing 122 detects, according to a site detection model 124, a plurality of target sites of observation 27 shown in the first ultrasound image 24B. In the example illustrated in FIG. 12, execution of the site detection processing 122 by the detection unit 62B results in the detection of an internal organ 27A, an internal organ 27B, and an internal organ 27C shown in the first ultrasound image 24B. In the example illustrated in FIG. 12, the site detection processing 122 is an example of “image recognition processing involving an AI approach” according to the technology of the present disclosure.

The site detection model 124 is a trained model for object detection according to an AI approach, and has been optimized by causing a neural network to undergo machine learning using second labeled training data. The second labeled training data is a plurality of data (that is, data of multiple frames) in which second example data and second ground truth data are associated with one another.

The second example data contains images corresponding to the ultrasound image 24. The second ground truth data contains ground truth data (that is, annotations) with respect to the second example data. As an example of the second ground truth data, annotations that can be used to identify sites corresponding to the target site of observation 27 in images corresponding to the ultrasound image 24 (for example, information indicating the names of target sites of observation 27, a plurality of coordinates that can be used to identify the position of each site corresponding to the target sites of observation 27, and information indicating a priority of sites) are used.

The detection unit 62B inputs the first ultrasound image 24B into the site detection model 124. This causes the site detection model 124 to detect a plurality of target sites of observation 27 shown in the inputted first ultrasound image 24B and output detection result information 126 indicating a detection result. The detection unit 62B acquires the detection result information 126 outputted from the site detection model 124.

The detection result information 126 includes site information 128 for each target site of observation 27 detected. The site information 128 includes site identification information 128A, position identification information 128B, and priority information 128C. The site identification information 128A is information synonymous with the site identification information 94A described in the embodiment above, and the position identification information 128B is information synonymous with the position identification information 94B described in the embodiment above. The priority information 128C is information indicating a priority of the target sites of observation 27 detected (for example, a predefined priority for each of the internal organ 27A, the internal organ 27B, and the internal organ 27C).

The measurement unit 62C performs various processing on each of the plurality of target sites of observation 27 detected from the first ultrasound image 24B, on the basis of the detection result information 126 acquired by the detection unit 62B.

For example, the measurement unit 62C refers to the position identification information 128B to identify the internal organs 27A to 27C from the first ultrasound image 24B to be processed by the site detection processing 122. For convenience, the description herein gives the example of the internal organs 27A to 27C, but this is merely one example, and the technology of the present disclosure is also achieved when applied to massive lesions (for example, masses or tumors) or vessels (for example, bile ducts or pancreatic ducts) instead of the internal organs 27A, 27B, and/or 27C.

The measurement unit 62C also refers to the site identification information 128A to identify a range 95 corresponding to each of the internal organ 27A, the internal organ 27B, and the internal organ 27C for each of the internal organ 27A, the internal organ 27B, and the internal organ 27C identified from the first ultrasound image 24B. These ranges 95 are defined in a similar manner to the embodiment above.

The measurement unit 62C measures the size 98 of each of the respective ranges 95 of the internal organ 27A, the internal organ 27B, and internal organ 27C, and adds size information 102 to each of the internal organ 27A, the internal organ 27B, and the internal organ 27C, in a similar manner to the embodiment above.

The measurement unit 62C generates calipers 96 for each of the internal organ 27A, the internal organ 27B, and the internal organ 27C, and adds caliper drawing information 100 to each of the internal organ 27A, the internal organ 27B, and the internal organ 27C, in a similar manner to the embodiment above.

Additionally, the measurement unit 62C refers to the detection result information 126 to add priority information 128C to each of the internal organ 27A, the internal organ 27B, and the internal organ 27C.

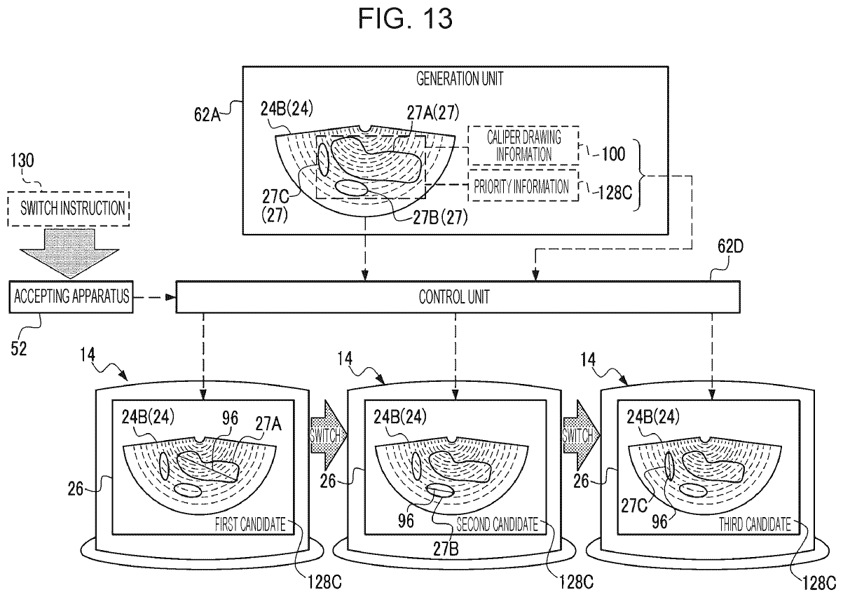

As illustrated by way of example in FIG. 13, the control unit 62D displays the first ultrasound image 24B on the screen 26, in a manner similar to the embodiment above. Within the first ultrasound image 24B, calipers 96 are displayed sequentially for each of the target sites of observation 27 on the basis of the caliper drawing information 100 applied to each of the target sites of observation 27. The calipers 96 for each of the target sites of observation 27 are displayed sequentially according to the priority indicated by the priority information 128C applied to the target site of observation 27 to which the calipers 96 are applied.

At the time of displaying the calipers 96 for each of the target sites of observation 27 on the screen 26, the priority information 128C is displayed on the screen 26 as information (in the example illustrated in FIG. 13, text information indicating “First Candidate”, “Second Candidate”, and “Third Candidate”) that can be used to identify the priority of the target site of observation 27 to which the displayed calipers 96 are applied. Since the range 95 is applied one at a time to the target sites of observation 27, the priority information 128C can also be thought of as information that can be used to identify the priority of the ranges 95. By displaying the priority information 128C on the screen 26 in this way, the physician 20 visually recognizes the priority of the range 95 defined by the calipers 96 displayed on the screen 26 (in other words, the priority of the target site of observation 27 to which the calipers 96 are applied).

While the first ultrasound image 24B is being displayed on the screen 26, the accepting apparatus 52 accepts a switch instruction 130, which is an instruction for switching the display of the calipers 96. In response to the switch instruction 130 accepted by the accepting apparatus 52, the control unit 62D switches the calipers 96 displayed on the screen 26 in an order (for example, ascending order or descending order) corresponding to the priority of the target site of observation 27 to which the calipers 96 are applied.

In the example illustrated in FIG. 13, the display is switched in the following order every time the accepting apparatus 52 accepts the switch instruction 130: the display of the calipers 96 applied to the internal organ 27A, the display of the calipers 96 applied to the internal organ 27B, and the display of the calipers 96 applied to the internal organ 27C. Also, the switching of the display of the calipers 96 is cyclic switching. For example, if the accepting apparatus 52 accepts the switch instruction 130 while the calipers 96 applied to the internal organ 27C are being displayed, the display is switched to the display of the calipers 96 applied to the internal organ 27A. In the example illustrated in FIG. 13, the switch instruction 130 is an example of a “fifth instruction” according to the technology of the present disclosure.

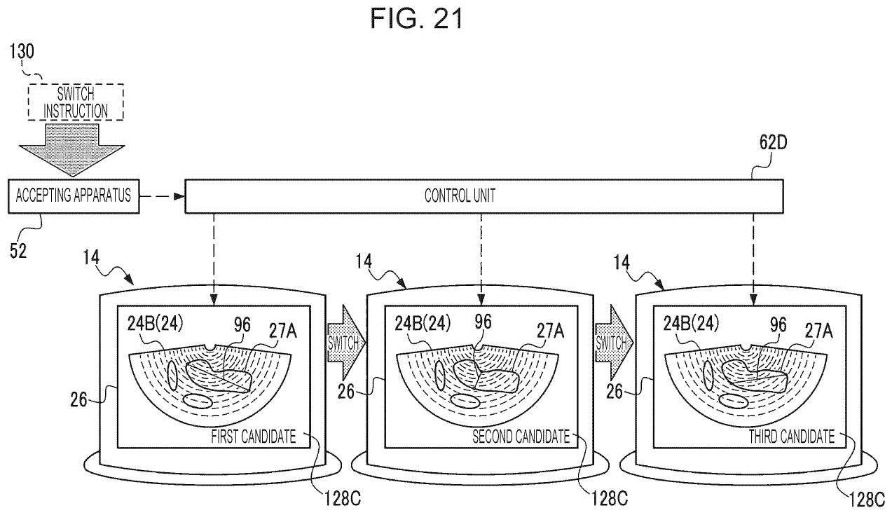

Although the above gives an example in which single calipers 96 are applied to the target site of observation 27, this is merely one example. For instance, as illustrated in FIG. 21, a plurality of calipers 96 may also be applied to a target site of observation 27 (in the example illustrated in FIG. 21, the internal organ 27A). In this case, a priority may be applied to each of the calipers 96, and the display of the calipers 96 may be switched according to the switch instruction 130 in an order defined according to the priority. The display of the calipers 96 may also be switched randomly, irrespective of priority.

In the example illustrated in FIG. 21, the display of a plurality of calipers 96 applied to the internal organ 27A is switched, but this is merely one example, and the display of a plurality of calipers 96 may also be switched in a similar manner across a plurality of target sites of observation 27 (for example, the internal organs 27A to 27C). In this case, for example, the display of the plurality of calipers 96 applied to each of the target sites of observation 27 may be switched so as to proceed from a high-priority target site of observation 27 to a low-priority target site of observation 27 among the plurality of target sites of observation 27.

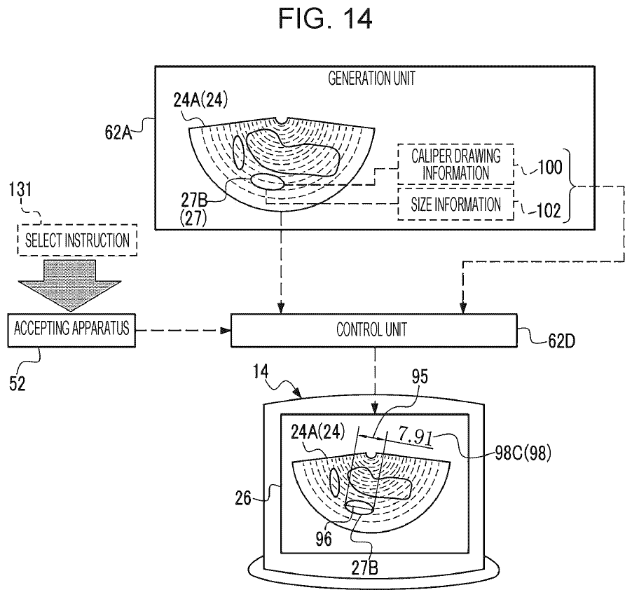

As illustrated by way of example in FIG. 14, the accepting apparatus 52 accepts a select instruction 131. The select instruction 131 selects the range 95 defined by the calipers 96 displayed on the screen 26. When the accepting apparatus 52 accepts the select instruction 131 while the calipers 96 are being displayed on the screen 26, the control unit 62D selects the range 95 defined by the calipers 96 currently being displayed on the screen 26. The control unit 62D acquires the size information 102 corresponding to the selected range 95 and outputs the acquired size information 102 to the display apparatus 14. This causes a third size 98C to be displayed on the screen 26, in correspondence with the range 95 defined by the calipers 96 on the screen 26. The third size 98C refers to the size 98 of the range 95 selected according to the select instruction 131.

In the example illustrated in FIG. 14, the size information 102 applied to the internal organ 27B is acquired by the control unit 62D and outputted to the display apparatus 14, and the size 98 of the range 95 defined by the calipers 96 applied to the internal organ 27B is displayed as the third size 98C on the screen 26.

In the example illustrated in FIG. 14, the select instruction 131 is an example of a “fourth instruction” according to the technology of the present disclosure, the size information 102 applied to the internal organ 27B is an example of “third size information” according to the technology of the present disclosure, and the third size 98C is an example of a “third size” according to the technology of the present disclosure.

In this way, in the case where a plurality of ranges 95 are identified through execution of the site detection processing 122 on the first ultrasound image 24B and the first ultrasound image 24B is displayed on the screen 26, the third size 98C of the range 95 selected according to a given select instruction 131 is displayed on the screen 26, which can enable the physician 20 observing the first ultrasound image 24B to ascertain the size 98 of the range 95 intended by the physician 20.

In the examples illustrated in FIGS. 13 and 14, the calipers 96 are displayed on the screen 26 according to the switch instruction 130, in an order corresponding to the priority indicated by the priority information 128C, and the size 98 of the range 95 selected according to the select instruction 131 is displayed as the third size 98C on the screen 26. This can enable the physician 20 observing the first ultrasound image 24B to quickly ascertain the size 98 of the range 95 intended by the physician 20.

In the example illustrated in FIG. 13, the display of the calipers 96 is switched according to the switch instruction 130, but the technology of the present disclosure is not limited thereto, and a plurality of calipers 96 may also be listed on the screen 26 in such a way that the priority can be identified. In this case, single calipers 96 may simply be selected according to the select instruction 131 among the listed plurality of calipers 96, and the size 98 of the range 95 defined by the selected calipers 96 may be displayed as the third size 98C on the screen 26.

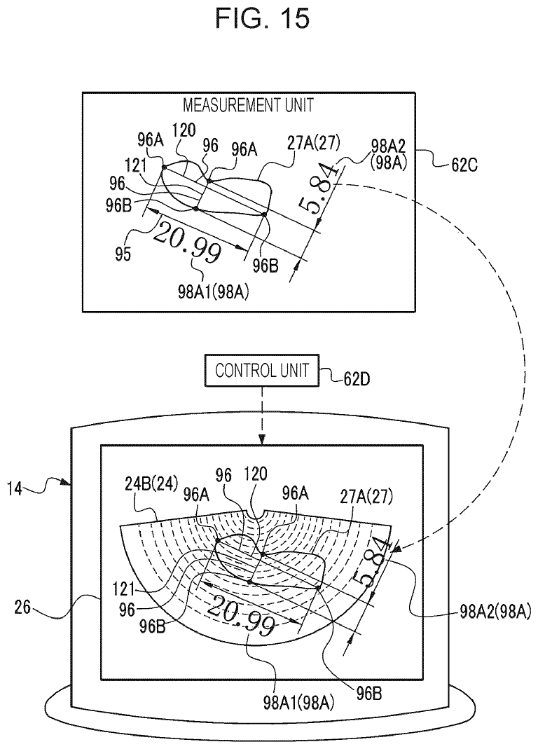

In the embodiment above, a maximum length of a cross section of the internal organ 27A is illustrated as an example of the first size 98A of the internal organ 27A shown in the first ultrasound image 24B, but the technology of the present disclosure is not limited thereto. For example, as illustrated in FIGS. 15 to 18, an ultrasound image 24 satisfying the following condition may be obtained as the first ultrasound image 24B: the size 98 of the target site of observation 27 (for example, the internal organ 27A) detected by the detection unit 62B is measured according to a measurement method appropriate for the target site of observation 27. The size 98 of the target site of observation 27 (for example, the internal organ 27A) shown in the first ultrasound image 24B may then be used as the first size 98A.

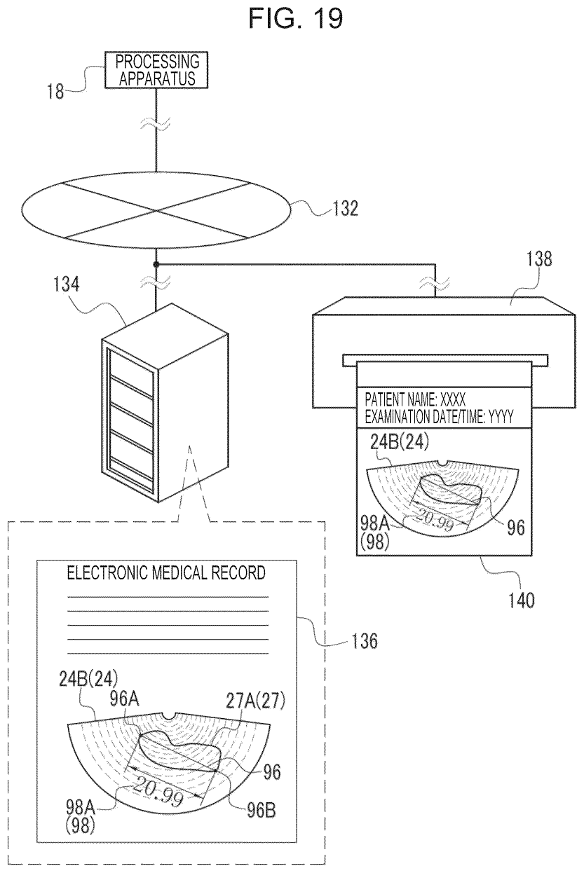

A plurality of ultrasound images 24 may also be displayed on the screen 26, outputted to an external apparatus 134, outputted to a printer 138, and/or saved in an electronic medical record 136 (see FIG. 19). In each of the plurality of ultrasound images 24, the size 98 of the target site of observation 27 (for example, the internal organ 27A) detected by the detection unit 62B is measured according to a measurement method appropriate for the target site of observation 27.

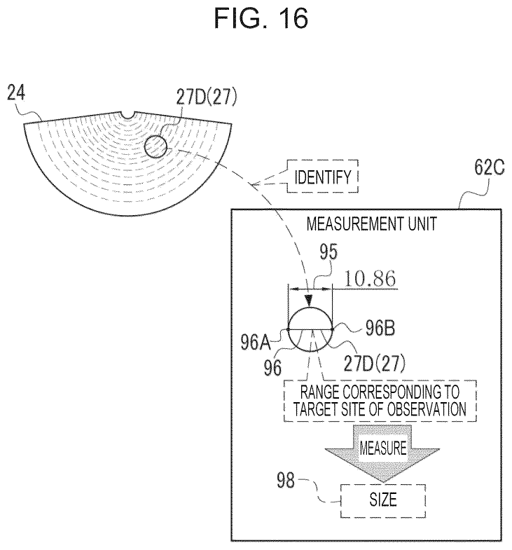

The measurement method in this case may be, for example, one or more measurement methods including a first measurement method and/or a second measurement method. As illustrated in FIG. 15, the first measurement method may be, for example, a method for measuring the target site of observation 27 (in the example illustrated in FIG. 15, the internal organ 27A) in multiple directions (in the example illustrated in FIG. 15, two directions). The multiple directions may also be three or more directions. The second measurement method may be, for example, a method for measuring the target site of observation 27 in one direction. Examples of methods for measuring in one direction include the method for measuring a maximum length of a cross section in a similar manner to the embodiment above, as well as the methods for measuring in one direction defined as illustrated in FIGS. 16 to 18.

As illustrated by way of example in FIG. 15, the measurement unit 62C may measure lengths 98A1 and 98A2 in two intersecting directions as the first size 98A. The length 98A1 is the length of a major axis 120 of a cross section of the internal organ 27A, and the length 98A2 is the length of a minor axis 121 of the cross section of the internal organ 27A. Calipers 96 are applied to each of the major axis 120 and the minor axis 121 in a manner similar to the embodiment above, and changing the geometric properties of the calipers 96 in a manner similar to the example illustrated in FIG. 11 also changes the lengths 98A1 and 98A2 measured by the measurement unit 62C.

The lengths 98A1 and 98A2 measured by the measurement unit 62C are displayed on the screen 26 by the control unit 62D, in a manner similar to the embodiment above. This enables the physician 20 observing the first ultrasound image 24B to ascertain the size 98 of the cross section of the internal organ 27A as the length 98A1 of the major axis 120 and the length 98A2 of the minor axis 121 in the cross section of the internal organ 27A.

Although the length 98A1 of the major axis 120 and the length 98A2 of the minor axis 121 are illustrated as an example herein, the technology of the present disclosure is also achieved with the length 98A1 of the major axis 120 or the length 98A2 of the minor axis 121. Also, instead of the internal organ 27A, the lengths of the major axis and/or minor axis of the internal organ 27B or the internal organ 27C may also be measured and displayed on the screen 26, and the lengths of the major axis and/or minor axis of a massive lesion may also be measured and displayed on the screen 26.

Although the lengths 98A1 and 98A2 in two intersecting directions are illustrated as an example of the first size 98A herein, this is merely one example, and three or more intersecting lengths may also be measured and displayed on the screen 26 as the first size 98A.

The embodiment above gives an example in which the measurement unit 62C measures the size 98 of the internal organ 27A shown in the ultrasound image 24, but this is merely one example. For instance, as illustrated in FIG. 16, the measurement unit 62C may also measure the size 98 of a vessel 27D. The vessel 27D may be, for example, a blood vessel, a lymphatic vessel, a bile duct, a pancreatic duct, or a medical artificial duct. The vessel 27D is an example of a “vessel” according to the technology of the present disclosure.

The measurement unit 62C identifies the vessel 27D as the target site of observation 27 from the ultrasound image 24 and sets a range 95 corresponding to the identified vessel 27D with respect to the vessel 27D, in a manner similar to the embodiment above. The measurement unit 62C also generates calipers 96 defining the range 95 with respect to the vessel 27D, in a manner similar to the embodiment above.

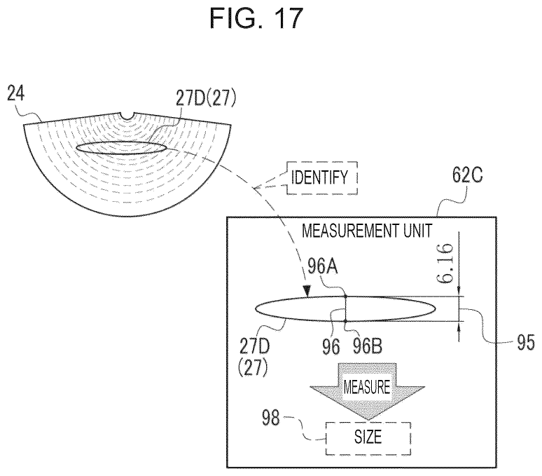

The measurement unit 62C measures the size 98 of the range 95 corresponding to the vessel 27D. In this case, the length of the radial range of the vessel 27D is measured as the size 98. In the case where the vessel 27D shown in the ultrasound image 24 is circular in a transverse sectional view, the diameter of the vessel 27D is measured as the size 98. As another example, as illustrated in FIG. 17, in the case where the vessel 27D shown in the ultrasound image 24 is ellipsoidal in a longitudinal sectional view, the length of the minor axis of the vessel 27D is measured as the size 98. In other words, a maximum length of a cross section of the vessel 27D shown in the ultrasound image 24 is measured as the size 98. The measured size 98 is displayed on the screen 26, in correspondence with the range 95 of the vessel 27D shown in the ultrasound image 24. This enables the physician 20 observing the ultrasound image 24 (for example, the first ultrasound image 24B) to ascertain the maximum length of the cross section of the vessel 27D shown in the ultrasound image 24.

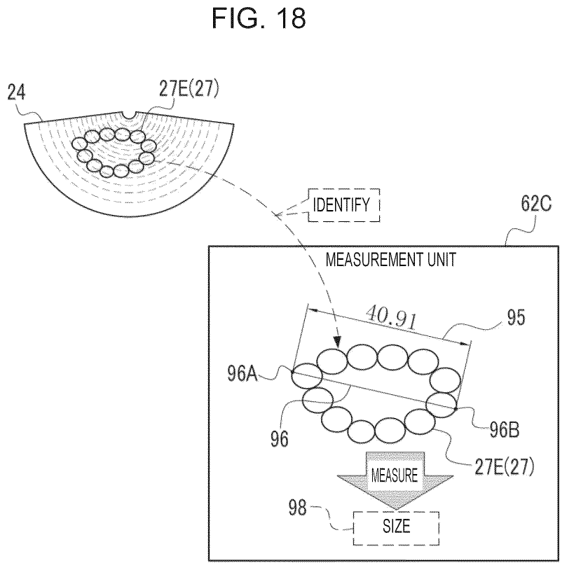

In the examples illustrated in FIGS. 16 and 17, the measurement unit 62C measures the size 98 of the vessel 27D shown in the ultrasound image 24, but as illustrated in FIG. 18, for example, the measurement unit 62C may also measure the size 98 of an annular lesion 27E shown in the ultrasound image 24. The annular lesion 27E may be, for example, a multivesicular lesion (for example, a multilocular cystic mass or a polycystic ovary). The annular lesion 27E is an example of an “annular lesion” according to the technology of the present disclosure.

The measurement unit 62C identifies the annular lesion 27E as the target site of observation 27 from the ultrasound image 24 and sets a range 95 corresponding to the identified annular lesion 27E with respect to the annular lesion 27E, in a manner similar to the embodiment above. The measurement unit 62C also generates calipers 96 defining the range 95 with respect to the annular lesion 27E, in a manner similar to the embodiment above.

The measurement unit 62C measures the size 98 of the range 95 corresponding to the annular lesion 27E. For example, the measurement unit 62C measures, as the size 98 of the range 95 corresponding to the annular lesion 27E, the size 98 of the range from one end to the other end of a line segment traversing the outline of the cross section of the annular lesion 27E shown in the ultrasound image 24. The measured size 98 is displayed on the screen 26, in correspondence with the range 95 of the annular lesion 27E shown in the ultrasound image 24. This enables the physician 20 observing the ultrasound image 24 (for example, the first ultrasound image 24B) to ascertain the size 98 of the range from one end to the other end of the line segment traversing the outline of the cross section of the annular lesion 27E shown in the ultrasound image 24.

The embodiment above gives an example in which the ultrasound image 24, the calipers 96, and the size 98 are displayed on the screen 26 through execution of the medical assistance processing by the processor 62 of the processing apparatus 18, but the technology of the present disclosure is not limited thereto.

For example, as illustrated in FIG. 19, in the case where the processing apparatus 18 is connected to an external apparatus 134 (for example, a server or a personal computer for managing electronic medical records 136 and/or images) over a network 132 such as a LAN or a WAN, the ultrasound image 24, the caliper drawing information 100, the size information 102 (for example, the first size information 110), and/or related information that is related to the size information 102 (for example, the first size information 110) may be saved in the external apparatus 134 and/or saved in an electronic medical record 136. The related information that is related to the size information 102 may be, for example, information (for example, a site name or coordinates) that can be used to identify the position of the target site of observation 27 of which the size 98 is measured, or information (for example, a site name or coordinates) that can be used to identify the current position of the ultrasound probe 38 that emitted the ultrasonic wave for obtaining the ultrasound image 24 showing the target site of observation 27 of which the size 98 is measured.

In this way, in the example illustrated in FIG. 19, the ultrasound image 24, the caliper drawing information 100, the size information 102, and/or related information that is related to the size information 102 are saved in the external apparatus 134, and/or saved in an electronic medical record 136. This can prevent the loss of the ultrasound image 24, the caliper drawing information 100, the size information 102, and/or the related information that is related to the size information 102.

The size information 102 and/or the related information that is related to the size information 102 may also be outputted as audio by an audio playback apparatus. The related information that is related to the size information 102 may also be displayed on the screen 26.

In the case where the processing apparatus 18 is connected to the printer 138 over the network 132, the ultrasound image 24, the caliper drawing information 100, the size information 102, and/or the related information that is related to the size information 102 may also be outputted to the printer 138. In this case, for example, the printer 138 prints the ultrasound image 24, the calipers 96, the size 98, and/or the related information that is related to the size information 102 onto a recording medium 140 (paper, for example).

The embodiment above gives an example in which the timespan 104 is set on the basis of the freeze image 24A, but this is merely one example, and the timespan 104 may also be designated according to an instruction accepted by the accepting apparatus 52 or any of various conditions (for example, the site shown in the ultrasound image 24 or the display appearance of the ultrasound image 24), irrespectively of the freeze image 24A. Alternatively, instead of designating the timespan 104, a plurality of ultrasound images 24 in a time series may be designated.

The embodiment above is described using an example in which the length of the range 95 is measured as the size 98, but this is merely one example, and the area and/or volume of a range corresponding to the target site of observation 27 may also be measured as the size 98. In other words, the size 98 is a concept that encompasses area and volume in addition to length.

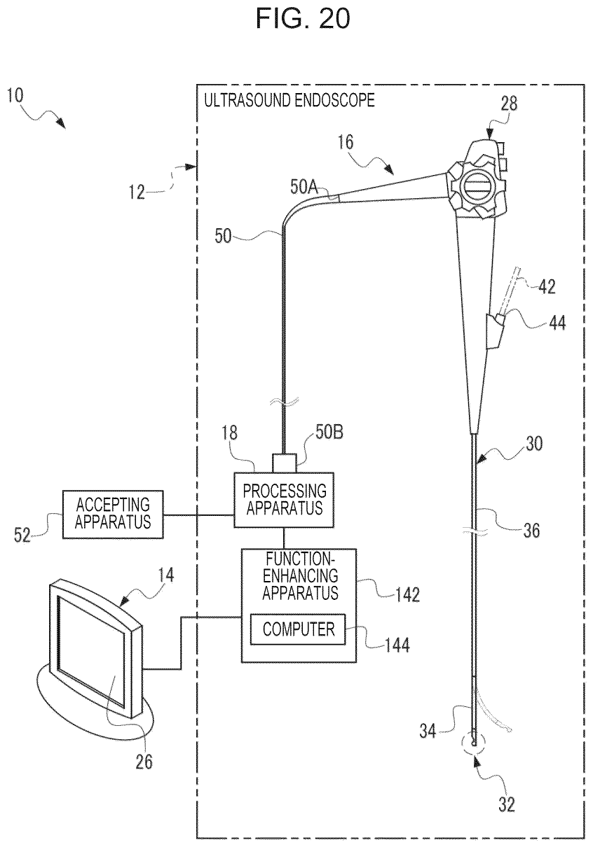

The embodiment above is described using an example in which the medical assistance processing is executed by the computer 54 of the processing apparatus 18, but the technology of the present disclosure is not limited thereto. For example, as illustrated in FIG. 20, the medical assistance processing may also be executed by a computer 144 (for example, a computer having a similar hardware configuration to the computer 54) of a function-enhancing apparatus 142, which is an auxiliary processing apparatus used to enhance the functions of the processing apparatus 18. The processing apparatus 18 and the display apparatus 14 are connected to the function-enhancing apparatus 142. The function-enhancing apparatus 142 executes the medical assistance processing under control by the processing apparatus 18, and outputs the ultrasound image 24, the caliper drawing information 100, the size information 102, and/or the related information that is related to the size information 102 to the display apparatus 14 and/or the like. This causes the ultrasound image 24, the calipers 96, the size 98, and/or the related information that is related to the size information 102 to be displayed on the screen 26 of the display apparatus 14.

The embodiment above gives an example in which the size 98 is measured by the measurement unit 62C in each ultrasound image 24 generated by the generation unit 62A, but the technology of the present disclosure is not limited thereto. For example, the size 98 may also be measured by the measurement unit 62C with respect to the ultrasound image 24 in one or more frames within a timespan (for example, the timespan 104) designated by the physician 20, and the size 98 may also be measured by the measurement unit 62C at intervals of a number of frames designated by the physician 20. The same applies to the site detection processing 93 and/or 122.

The embodiment above is described using an example in which the medical assistance program 76 is stored in the NVM 66, but the technology of the present disclosure is not limited thereto. For example, the medical assistance program 76 may also be stored in a computer-readable storage medium such as an SSD or USB memory. The storage medium is a stationary or portable non-transitory storage medium. The medical assistance program 76 stored in the storage medium is installed in the computer 54. The processor 62 executes the medical assistance processing according to the medical assistance program 76.

In the embodiment above, the computer 54 is illustrated by way of example, but the technology of the present disclosure is not limited thereto, and a device including an ASIC, an FPGA, and/or a PLD may also be applied in place of the computer 54. A combination of a hardware configuration and a software configuration may also be used in place of the computer 54.

The various types of processors indicated below can be used as hardware resources to execute the medical assistance processing described in the embodiment above. The processor may be, for example, a general-purpose processor that executes software, namely a program, to thereby function as hardware resources to execute the medical assistance processing. The processor may also be, for example, a special-purpose electronic circuit such as an FPGA, a PLD, or an ASIC, that is, a processor having a specially designed circuit configuration for executing specific processing. Any of these processors has a built-in or connected memory, and any of these processors uses the memory to execute the medical assistance processing.

The hardware resources to execute the medical assistance processing may be formed from one of these various types of processors, or may be formed from a combination of two or more processors of the same or different types (such as a combination of multiple FPGAS, or a combination of a processor and an FPGA). The hardware resources to execute the medical assistance processing may also be a single processor.

As a first example of a configuration using a single processor, a combination of one or more processors and software are used to form a single processor, and this processor functions as the hardware resources to execute the medical assistance processing. A second example is to use processor in which the functions of the entire system, including multiple hardware resources to execute the medical assistance processing, are realized by a single IC chip, as typified by an SoC. In this way, the medical assistance processing is realized by using one or more of the various types of processors above as hardware resources.

Furthermore, an electronic circuit combining circuit elements such as semiconductor elements can be used more specifically as the hardware structure of these various types of processors. Also, the medical assistance processing above is merely one example. Needless to say, unnecessary steps may be deleted, new steps may be added, and the processing sequence may be rearranged, insofar as the result does not depart from the gist of the technology of the present disclosure.

The descriptions and illustrations given above are detailed descriptions of portions related to the technology of the present disclosure, and are nothing more than examples of the technology of the present disclosure. For example, the above descriptions pertaining to configuration, function, action, and effect are descriptions pertaining to one example of the configuration, function, action, and effect of portions related to the technology of the present disclosure. Needless to say, unnecessary portions may be deleted and new elements may be added or substituted with respect to the descriptions and illustrations given above, insofar as the result does not depart from the gist of the technology of the present disclosure. Also, to avoid confusion and to facilitate understanding of the portions related to the technology of the present disclosure, in the descriptions and illustrations given above, description is omitted in regard to common technical knowledge and the like that does not require particular explanation to enable implementation of the technology of the present disclosure.

In this specification, “A and/or B” is synonymous with “at least one of A or B”. That is, “A and/or B” means that: A only is a possibility; B only is a possibility; and a combination of A and B is a possibility. Also, in this specification, the same way of thinking as for “A and/or B” also applies when three or more matters are expressively linked using “and/or”.

All documents, patent applications, and technical standards mentioned in this specification are incorporated by reference herein to the same extent that individual documents, patent applications, and technical standards are specifically and individually noted as being incorporated by reference.

What is claimed is:

1. A medical assistance apparatus comprising a processor
the processor is configured to:
display, on a screen, a first ultrasound image among a plurality of ultrasound images in a time series, the ultrasound image showing a target site of observation and satisfying a predetermined condition;
output first size information indicating a first size;
detect the target site of observation by performing image recognition processing on the ultrasound image; and
measure the target site of observation according to a measurement method appropriate for the detected target site of observation, wherein
the first size is a size of the target site of observation shown in the first ultrasound image,
the predetermined condition includes a condition stipulating that a length of the target site of observation is equal to or greater than a reference value,
the measurement method includes a first measurement method and/or a second measurement method,
the first measurement method is a method for measuring the target site of observation in one direction, and
the second measurement method is a method for measuring the target site of observation in multiple directions.

2. The medical assistance apparatus according to claim 1, wherein
the outputting of the first size information includes displaying the first size on the screen.

3. The medical assistance apparatus according to claim 1, wherein
the predetermined condition includes a condition stipulating an image which is among the plurality of ultrasound images and in which the target site of observation is detected and the size of the detected target site of observation is measured.

4. The medical assistance apparatus according to claim 1, wherein
the predetermined condition includes a condition stipulating an image which is among the plurality of ultrasound images and in which a specific target site of observation is detected among a plurality of target sites of observation and the size of the specific target site of observation is measured.

5. The medical assistance apparatus according to claim 1, wherein
the processor is configured to output a plurality of ultrasound images which are among the plurality of ultrasound images and in which the target site of observation is detected and the size of the target site of observation is measured.

6. The medical assistance apparatus according to claim 1, wherein
the predetermined condition includes a condition stipulating that a length of the target site of observation is a maximum value or a mode.

7. The medical assistance apparatus according to claim 1, wherein
in a case in which the target site of observation is a vessel,
the length is the length in the radial direction of the vessel.

8. The medical assistance apparatus according to claim 1, wherein
size information indicating the size of the target site of observation is applied to each of the ultrasound images, and
the processor is configured to output the size information applied to the first ultrasound image as the first size information.

9. The medical assistance apparatus according to claim 1, wherein
the first ultrasound image is an ultrasound image that satisfies the predetermined condition from among the ultrasound images in a timespan defined with respect to a selected ultrasound image that is selected from the plurality of ultrasound images according to a given first instruction.

10. The medical assistance apparatus according to claim 9, wherein
the selected ultrasound image is a freeze image, and
the freeze image is the ultrasound image displayed on the screen in a frozen state according to the first instruction in a situation in which the plurality of ultrasound images are being displayed as a dynamic image on the screen.

11. The medical assistance apparatus according to claim 9, wherein
the timespan is a timespan going back from the point in time when the selected ultrasound image is obtained.

12. The medical assistance apparatus according to claim 10, wherein
the length of the timespan is defined according to a given second instruction.

13. The medical assistance apparatus according to claim 1, wherein
the processor is configured to output second size information indicating a second size, and
the second size is a size of the target site of observation shown in a second ultrasound image different from the first ultrasound image among the plurality of ultrasound images.

14. The medical assistance apparatus according to claim 13, wherein
size information indicating the size of the target site of observation is applied to each of the ultrasound images, and
the processor is configured to output the size information applied to the second ultrasound image as the second size information.

15. The medical assistance apparatus according to claim 13, wherein
the outputting of the second size information includes displaying the second size on the screen.

16. The medical assistance apparatus according to claim 13, wherein
the processor is configured to output the first size information and the second size information in a distinguishable manner.

17. The medical assistance apparatus according to claim 1, wherein
the predetermined condition includes a condition stipulating an image which is among the plurality of ultrasound images and in which the target site of observation is detected and the size of the target site of observation is measured according to a measurement method appropriate for the detected target site of observation.

18. The medical assistance apparatus according to claim 1, wherein
the predetermined condition includes a condition stipulating an image which is among the plurality of ultrasound images and in which a specific target site of observation is detected among a plurality of target sites of observation and the size of the specific target site of observation is measured according to a measurement method appropriate for the specific target site of observation.

19. The medical assistance apparatus according to claim 1, wherein
the processor is configured to output a plurality of ultrasound images which are among the plurality of ultrasound images and in which the target site of observation is detected and the size of the target site of observation is measured according to a measurement method appropriate for the target site of observation.

20. The medical assistance apparatus according to claim 19, wherein
the measurement method includes a first measurement method and/or a second measurement method,
the first measurement method is a method for measuring the target site of observation in one direction, and
the second measurement method is a method for measuring the target site of observation in multiple directions.

21. The medical assistance apparatus according to claim 1, wherein
the first size is a size of a range corresponding to the target site of observation.

22. The medical assistance apparatus according to claim 21, wherein
calipers defining the range are displayed on the screen.

23. The medical assistance apparatus according to claim 22, wherein
a geometric property of the calipers is changed according to a given third instruction, and
the range is changed in association with the change in the geometric property.

24. The medical assistance apparatus according to claim 21, wherein
while the first ultrasound image is being displayed on the screen,
the processor is configured to output third size information indicating a third size of the range selected according to a given fourth instruction among a plurality of ranges.

25. The medical assistance apparatus according to claim 24, wherein
the ranges are assigned a priority, and
calipers indicating the ranges are displayed in a state allowing for identification of the priority.

26. The medical assistance apparatus according to claim 25, wherein
the calipers are displayed on the screen in an order corresponding to the priority, and
the calipers to be displayed on the screen are switched according to a given fifth instruction.

27. The medical assistance apparatus according to claim 1, wherein
the first size information and/or related information that is related to the first size information are saved in an external apparatus and/or medical record.

28. The medical assistance apparatus according to claim 1, wherein
the ultrasound image is an endoscopic ultrasound image.

29. An ultrasound endoscope comprising:
the medical assistance apparatus according to claim 1; and
an ultrasound probe that, when inserted into a body, emits an ultrasonic wave inside the body and receives a reflected wave of the ultrasonic wave, wherein
the ultrasound image is generated on a basis of the reflected wave.

30. A medical assistance method comprising:
displaying, on a screen, a first ultrasound image among a plurality of ultrasound images in a time series, the ultrasound image showing a target site of observation and satisfying a predetermined condition;
outputting first size information indicating a first size;
detecting the target site of observation by performing image recognition processing on the ultrasound image; and
measuring the target site of observation according to a measurement method appropriate for the detected target site of observation, wherein
the first size is a size of the target site of observation shown in the first ultrasound image,
the predetermined condition includes a condition stipulating that a length of the target site of observation is equal to or greater than a reference value,
the measurement method includes a first measurement method and/or a second measurement method,
the first measurement method is a method for measuring the target site of observation in one direction, and
the second measurement method is a method for measuring the target site of observation in multiple directions.

31. A non-transitory computer-readable storage medium storing a program executable by a computer to execute a process comprising:
displaying, on a screen, a first ultrasound image among a plurality of ultrasound images in a time series, the ultrasound image showing a target site of observation and satisfying a predetermined condition;
outputting first size information indicating a first size;
detecting the target site of observation by performing image recognition processing on the ultrasound image; and
measuring the target site of observation according to a measurement method appropriate for the detected target site of observation, wherein
the first size is a size of the target site of observation shown in the first ultrasound image,
the predetermined condition includes a condition stipulating that a length of the target site of observation is equal to or greater than a reference value,
the measurement method includes a first measurement method and/or a second measurement method,
the first measurement method is a method for measuring the target site of observation in one direction, and
the second measurement method is a method for measuring the target site of observation in multiple directions.
